(19) United States
(12) Patent Application Publication
(10) Pub. No.: US 2011/0189310 A1
(43) Pub. Date: Aug. 4, 2011

(54) SMALL MOLECULE MODULATORS OF PRONGF UPTAKE

(75) Inventor: Barbara L. Hempstead, New York, NY (US)

(73) Assignee: CORNELL UNIVERSITY, Ithaca, NY (US)

(21) Appl. No.: 13/122,483

(22) PCT Filed: Oct. 8, 2009
(86) PCT No.: PCT/US09/60019

§ 371 Date: Apr. 4, 2011

Related U.S. Application Data
(62) Division of application No. 61/103,799, filed on Oct. 8, 2008.

(51) Int. Cl. A61K 33/00; A61K 31/12; A61K 31/198; A61K 31/366; A61P 25/28; A61P 25/24; A61P 25/14; A61P 9/00; A61P 9/10

(52) U.S. Cl. 424/722; 514/689; 514/567; 514/460

(57) ABSTRACT

The present invention relates to a method of inhibiting cellular uptake of pro-nerve growth factor (proNGF) in a cell expressing neurotrophin p75 receptor in a mammal in need thereof. Such mammals include, for example, those suffering from neuropathological conditions. In another aspect, the invention relates to a method of promoting cellular uptake of proNGF in a cell expressing p75^{NTR} receptor in a mammal in need thereof. Such mammals include, for example, those suffering from conditions relating to human and other mammalian hair follicle cycling.

This application asserts priority to U.S. Provisional Application No. 61/103,799, filed Oct. 8, 2008, the contents of which are hereby incorporated by reference in its entirety.

The invention was made with funds from NY State Spinal Cord Injury Board.

### BACKGROUND OF THE INVENTION

Neurotrophins [nerve growth factor (NGF), brain derived neurotrophic factor (BDNF), neurotrophin-3 (NT-3), and neurotrophin-4/5 (NT-4/5)] are secreted proteins that have potent effects on neuronal survival, differentiation, and synaptic function (Chao, 2003; Huang and Reichardt, 2003; Teng and Hempstead, 2004; Lu et al., 2005) as well as effects in the cardiovascular system. Mature neurotrophins bind preferentially to tropomyosin-related kinase (Trk) receptors in conjunction with neurotrophin p75 receptor (p75^{NTR}) and other receptors.

Proneurotrophins, which contain an N-terminal domain proteolytically removed in “mature” forms, were originally considered to be inert precursor molecules to the mature forms. However, they have been found to have biological activity—they interact with p75^{NTR} and through their N-terminal domains, with the sorting receptor sortilin (Fahnestock et al., 2001; Harrington et al., 2004; Nykjaer et al., 2004; Teng et al., 2005; Volosin et al., 2006) and other receptors. See especially published PCT application WO2002096356A2.

Proneurotrophins have been found to be secreted by cells in vitro (Teng et al, 2005; Domeniconi et al, 2007); secreted in vivo in animal models of injury (Harrington et al., 2004) and in animals with normal brain function (Pang et al. 2004) and evidence of secretion has been found in post-mortem samples of the brains of human Alzheimer's patients (Peng et al., 2004 and Pedraza et al., 2005).

In particular, pro-nerve growth factor (proNGF), the precursor form of NGF, induces death of neurons and oligodendrocytes through p75^{NTR}, and its concomitant binding to p75^{NTR} and sortilin has been shown to activate death pathways (Lee et al., 2001; Beattie et al., 2002; Nykjaer et al., 2004; Domeniconi et al, 2007). For example, proNGF binding to p75^{NTR} can induce apoptosis after injury. This has been demonstrated experimentally in animal models of spinal cord injury (Harrington et al., 2004; Beattie et al 2002); animal models of seizure disorders such as epilepsy (Volosin et al., 2006; Volosin et al., 2008); and models of Alzheimers (Peng et al., 2004; Pedraza et al., 2005). Other recent evidence suggests that p75^{NTR} interacting with proteins such as proNGF might mediate cell death in various neuropathological conditions, including Alzheimer's disease, spinal cord trauma, axotomy, and retinal dystrophy (Nykjaer et al., 2005). Accordingly, inhibiting cellular uptake of proNGF in a cell expressing p75^{NTR} receptor would be beneficial for cell survival.

In other signal-transduction pathways, p75^{NTR} signaling through phosphatidylinositol 3-kinase AKT (PI3K/AKT) or interleukin-1 receptor-associated kinase/nuclear factor {kappa}B (IRAK/NF{kappa}B) promotes survival (Carter et al., 1996; Roux et al., 2001; Mamidipudi et al., 2002; Reddypalli et al., 2005). For example, expression, secretion, and signalling by neurotrophins, proneutrophins, and their receptors has been found to be important to development and maintenance of neurons and hair follicles involved in hearing (Sato et al, 2006).

In addition, expression, secretion, and signalling by neurotrophins, proneutrophins, and their receptors, particularly proNGF, NGF, p75^{NTR} and trkA, have been found to cycle in conjunction with human and other mammalian hair follicle cycling, and to be active in skin immune cells, keratinocytes, melanocytes, sebaceous glands, and sweat glands (Adly et al, 2006; Bläsing H et al 2005; Peters et al, 2006a; Peters et al 2006b). Accordingly, promoting cellular uptake of proNGF in a cell expressing p75^{NTR} receptor would be beneficial for cell survival.

### SUMMARY OF THE INVENTION

The above objectives have been met by the present invention which provides, in one aspect, a method of inhibiting cellular uptake of pro-nerve growth factor in a cell expressing neurotrophin p75 receptor in a mammal in need thereof The method includes administering to the mammal an effective amount of any one or any combination of the following inhibiting compounds: 2′,2′-Bisepigallocatechin Monogallate; 2-Aminoethyldiphenyl Boronate; 2-Isopropyl-3-Methoxycinnamic Acid; 4,4′-diisothiocyanostilbene-2,2′-disulfonic acid Sodium Salt; 5-Chloroindole-2-Carboxylic Acid; 5-Fluoroindole-2-Carboxylic Acid; Acetohydroxamic Acid; Acetyl Tyrosine Ethyl Ester; Almotriptan; Almotriptan; Apramycin; Atorvastatin Calcium; Aurintricarboxylic Acid; Aurothioglucose; Butamben; Calcein; Crustecdysone; 2,3,4,6-tetrakis-O-(3-nitropropanoyl)-alpha-L-galactopyranose; Epicatechin Monogallate; Epigallocatechin-3-Monogallate; Glyburide; Hematein; Hinokitiol; Homosalate; Hydroquinone; Indole-2-Carboxylic Acid; Iopanic Acid; Irigenol; iriginol hexaaceatate; Levodopa; Lithium Citrate; Lovastatin; Meclizine Hydrochloride; Miglitol; N-(9-Fluorenylmethoxycarbonyl)-L-Leucine; Ononetin; Oxaprozin; Oxybenzone; Perillic Acid (−); Perindopril Erbumine; Prazosin Hydrochloride; Prenyletin; Quercetin Pentamethyl Ether; Ribostamycin Sulfate; Robustic Acid; Sparteine Sulfate; Sulconazole Nitrate; Sumaresinolic Acid; Suramin; Tannic Acid; Theaflavin; Theaflavin Digallate; or a pharmaceutically acceptable salt of any such compound, wherein the cellular uptake of proNGF is decreased at least about 60% than in the absence of the compound, when measured under same conditions.

In another aspect, the invention provides a method of promoting cellular uptake of proNGF in a cell expressing p75^{NTR} receptor in a mammal in need thereof. The method includes administering to the mammal an effective amount of any one or any combination of the following promoting compounds Trichlormethine; 6,7-Dihydroxyflavone; Tangeritin; 3′,4′-Dihydroxyflavone; Amsacrine Hydrochloride; Putrescine Dihydrochloride; Flunixin Meglumine; 3-Benzyloxy-4,4-Bisnor-8,11,13-Podocarpatriene; 2-Acetylpyrrole; Dantron; 2,3,4-Trihydroxy-4′-Ethoxybenzophenone; 8-Cyclopentyltheophylline; Aminocyclopropanecarboxylic Acid; 2′,3-Dihydroxy-4,4′,6′-Trimethoxychalcone; 3,5-Dinitrocatechol (Or-486); Aztreonam; Piceid; Rifaximin; 10-Hydroxycamptothecin; 5,7-Dihydroxyflavone; ANTIMYCIN A; Xanthoxylin; or a pharmaceutically acceptable salt of any such compound, wherein the cellular uptake of proNGF is increased at least about 180% than in the absence of the compound, when measured under same conditions.

### BRIEF DESCRIPTION OF THE FIGURES

Table 1 Names and structures of small molecule inhibitors of proNGF uptake with normalized percent of proNGF uptake.

Table 2 Names and structures of small molecule promoters of proNGF uptake with normalized percent of proNGF uptake

Table 3 Names of small molecule inhibitors of proNGF uptake with normalized percent of proNGF uptake from Table 1, and calculated decrease of cellular uptake of proNGF (i.e., 1 minus normalized percent of proNGF uptake from Table 1).

Table 4 Names of small molecule inhibitors of proNGF uptake with normalized percent of promotion from Table 2, sorted in order of ascending normalized percent of proNGF uptake.

FIG. 1: Pharmacological blockade of proNGF induced neuronal apoptosis. Cultured rat SCG neurons (7 DIV) were washed free of NGF and were treated or not with 10 ng/ml NGF or 5 ng/ml proNGF in the presence or absence of 10 μM Lithium Citrate or L-DOPA, or 20 μM neurotensin. (A) Neuronal apoptosis was assessed morphologically by DAPI and TuJ1 double staining and (B) quantitated as percentage of SCG neuron apoptosis. The numbers represent results from three independently conducted experiments. Error bars represent S.E.M.

FIG. 2: Images of normal human brain tissue and glioblastoma, demonstrating upregulation of p75 in the leading, invasive edge of glioblastoma.

FIG. 3: ProNGF and p75 are induced following cardiac ischemia. A) and B) show levels of p75 receptor expression on murine cardiac vessels subsequent (A) and prior (B) to occlusion of a major coronary artery; C) shows proNGF expression in cardiomyoctes subsequent to occlusion of a major coronary artery.

### DETAILED DESCRIPTION OF THE INVENTION

Method for Inhibiting Cellular Uptake of proNGF

In one aspect, the invention provides a method of inhibiting cellular uptake of proNGF in a cell expressing p75^{NTR} receptor in a mammal in need thereof The term “inhibiting” cellular uptake of proNGF in a cell expressing p75^{NTR} receptor refers to a decreased level in measurable cellular uptake of proNGF by a cell in a given assay in the presence of a compound, relative to the level of measurable cellular uptake in the absence of the compound, when tested under the same conditions.

Cellular uptake of proNGF is considered inhibited according to the invention if cellular uptake of proNGF is decreased at least about 60%, preferably at least about 70%, more preferably at least about 85%, even more preferably at least about 95%, and yet even more preferably at least about 99%; and most preferably decreased at 100% than in the absence of the compound (e.g. as measured by the cellular uptake of labeled proNGF), when measured under same conditions.

“Cellular uptake” of proNGF or “proNGF uptake” refers to internalization of proNGF into a cell, without implying a specific mechanism of uptake. Examples of mechanisms of cellular uptake include, but not limited to, specific or non-specific engulfment, pinocytosis, endocytosis, and/or phagocytosis, or cellular ingestion. A decrease in cellular uptake of proNGF is correlated with a decrease in total cellular proNGF. Conversely, an increase in cellular uptake of proNGF is correlated with an increase in total cellular proNGF.

In accordance with the invention, cellular uptake of proNGF is inhibited in any cell expressing p75^{NTR} receptor. Examples of cells expressing p75^{NTR} receptor include neural cells. Any neural cell may be involved in the methods of the invention. As used herein, the phrase “neural cell” includes nerve cells (i.e., neurons, e.g., uni-, bi-, or mulipolar neurons) and their precursors and glial cells (e.g., glia such as astrocytes, oligodendrocytes, ependymal cells, radial glia, Schwann cells, Satellite cells, and microglia) and their precursors. Microglia are specialized macrophages capable of phagocytosis that protect neurons of the central nervous system. The term “precursor” refers to cells that are capable of developing into a specific cell type. For example, a neural cell precursor is a cell which is capable of developing into a mature neural cell (i.e., a cell having the characteristic morphology and function of a neural cell).

Additional examples of expressing p75^{NTR} receptor include cells of the central nervous system (CNS) or peripheral nervous system (PNS), which include neurons, glial cells, Schwann cells, astrocytes, oligodendrocytes, microglia cells, endothelial cells, immune cells (e.g., macrophages, T cells, B cells, and neutrophils), etc.

Further examples of cells expressing p75^{NTR} receptor include an endothelial cell, smooth muscle cell, cardiomyocyte, pericyte, skin cell, hair follicle cell, skin immune system cell, keratinocyte, melanocyte, sebaceous gland cell, and sweat gland cell. In one embodiment, the cell is in a mammal, preferably a human.

The p75 neurotrophin receptor (p75^{NTR}) is a member of the tumor necrosis factor receptor (TNRF) family. It is a 75 kDa cell-surface receptor glycoprotein that binds with similar affinity to the neurotrophin family (BDNF, NT-3, and NT-4/5) of growth factors. p75^{NTR} is a mammalian p75^{NTR}, preferably human p75^{NTR}.

Pro-nerve growth factor (proNGF) refers to a precursor molecule in the synthesis of nerve growth factor (NGF). ProNGF undergoes processing (i.e., cleavage) to provide mature NGF. The proNGF is mammalian, preferably human proNGF.

In one embodiment, the method includes administering to the mammal in need thereof an effective amount of any one or any combination of the following inhibiting compounds: 2′,2′-Bisepigallocatechin Monogallate; 2-Aminoethyldiphenyl Boronate; 2-Isopropyl-3-Methoxycinnamic Acid; 4,4′-diisothiocyanostilbene-2,2′-disulfonic acid Sodium Salt; 5-Chloroindole-2-Carboxylic Acid; 5-Fluoroindole-2-Carboxylic Acid; Acetohydroxamic Acid; Acetyl Tyrosine Ethyl Ester; Almotriptan; Almotriptan; Apramycin; Atorvastatin Calcium; Aurintricarboxylic Acid; Aurothioglucose; Butamben; Calcein; Crustecdysone; 2,3,4,6-tetrakis-O-(3-nitropropanoyl)-alpha-L-galactopyranose; Epicatechin Monogallate; Epigallocatechin-3-Monogallate; Glyburide; Hematein; Hinokitiol; Homosalate; Hydroquinone; Indole-2-Carboxylic Acid; Iopanic Acid; Irigenol; iriginol hexaaceatate; Levodopa; Lithium Citrate; Lovastatin; Meclizine Hydrochloride; Miglitol; N-(9-Fluorenylmethoxycarbonyl)-L-Leucine; Ononetin; Oxaprozin; Oxybenzone; Perillic Acid (−); Perindopril Erbumine; Prazosin Hydrochloride; Prenyletin; Quercetin Pentamethyl Ether; Ribostamycin Sulfate; Robustic Acid; Sparteine Sulfate; Sulconazole Nitrate; Sumaresinolic Acid; Suramin; Tannic Acid; Theaflavin; Theaflavin Digallate; or a pharmaceutically acceptable salt of any such compound, wherein cellular uptake of proNGF is decreased at least about 60% than in the absence of the compound, when measured under same conditions.

An “inhibiting” compound refers to a compound that inhibits cellular uptake of proNGF in a cell expressing p75^{NTR} receptor, as described above, i.e., one that decreases the level of measurable cellular uptake of proNGF by a cell in a given assay in the presence of a compound, relative to the level of measurable cellular uptake in the absence of the compound, when tested under the same conditions.

In a further embodiment, the method includes administering to the mammal in need thereof an effective amount of any one or any combination of the following inhibiting compounds: Hydroquinone; Tannic Acid; Epigallocatechin-3-Monogallate; Aurintricarboxylic Acid; Sulconazole Nitrate; Levodopa; 2′,2′-Bisepigallocatechin Monogallate; 4,4′-diisothiocyanostilbene-2,2′-disulfonic acid Sodium Salt; Hematein; 5-Fluoroindole-2-Carboxylic Acid; Epicatechin Monogallate; Meclizine Hydrochloride; Theaflavin Digallate; Irigenol; Suramin; Atorvastatin Calcium; Indole-2-Carboxylic Acid; Iopanic Acid; N-(9-Fluorenylmethoxycarbonyl)-L-Leucine; Miglitol; Perillic Acid (−);Acetyl Tyrosine Ethyl Ester; Prenyletin; 2-Isopropyl-3-Methoxycinnamic Acid; Prazosin Hydrochloride; 5-Chloroindole-2-Carboxylic Acid; Theaflavin; Homosalate; Sumaresinolic Acid; Lovastatin; Robustic Acid; Glyburide; Oxybenzone; Calcein; 2,3,4,6-tetrakis-O-(3-nitropropanoyl)-alpha-L-galactopyranose; Sparteine Sulfate; iriginol hexaaceatate; 2-Aminoethyldiphenyl Boronate; Ribostamycin Sulfate; Hinokitiol; or a pharmaceutically acceptable salt of any such compound, wherein cellular uptake of proNGF is decreased at least about 70% than in the absence of the compound, when measured under same conditions.

In yet another embodiment, the method includes administering to the mammal in need thereof an effective amount of any one or a combination of the following inhibiting compounds: Hydroquinone; Tannic Acid; Epigallocatechin-3-Monogallate; Aurintricarboxylic Acid; Sulconazole Nitrate; Levodopa; 2′,2′-Bisepigallocatechin Monogallate; 4,4′-diisothiocyanostilbene-2,2′-disulfonic acid Sodium Salt; Hematein; 5-Fluoroindole-2-Carboxylic Acid; Epicatechin Monogallate; Meclizine Hydrochloride; Theaflavin Digallate; Irigenol; Suramin; or a pharmaceutically acceptable salt of any such compound, and wherein the cellular uptake of proNGF is decreased at least about 85% than in the absence of the compound, when measured under same conditions.

In a preferred embodiment, the method includes administering to the mammal in need thereof an effective amount of any one or a combination of the following inhibiting compounds: Levodopa; lithium, or a statin such as lovastatin or a pharmaceutically acceptable salt of any such compound, and wherein the cellular uptake of proNGF is decreased at least about 99% than in the absence of the compound, when measured under same conditions.

Method of Promoting Cellular Uptake of proNGF

In another aspect, the invention provides a method of promoting cellular uptake of proNGF in a cell expressing p75NTR receptor in a mammal in need thereof. The term “promoting” cellular uptake of proNGF in a cell expressing p75^{NTR} receptor refers to increasing measurable cellular uptake of proNGF by a cell in a given assay in the presence of a compound, relative to the level of measurable cellular uptake in the absence of the compound, when tested under the same conditions.

Cellular uptake of proNGF is considered promoted according to the invention if cellular uptake of proNGF is increased at least about 180%, preferably at least about 200%, more preferably at least about 300%, even more preferably at least about 400%, and yet even more preferably at least about 500%; and most preferably increased at 700%, or more than in the absence of the compound (e.g. as measured by the cellular uptake of labeled proNGF), when measured under same conditions.

The method includes administering to the mammal an effective amount of any one or any combination of the following promoting compounds: Trichlormethine; 6,7-Dihydroxyflavone; Tangeritin; 3′,4′-Dihydroxyflavone; Amsacrine Hydrochloride; Putrescine Dihydrochloride; Flunixin Meglumine; 3-Benzyloxy-4,4-Bisnor-8,11,13-Podocarpatriene; 2-Acetylpyrrole; Dantron; 2,3,4-Trihydroxy-4′-Ethoxybenzophenone; 8-Cyclopentyltheophylline; Aminocyclopropanecarboxylic Acid; 2′,3-Dihydroxy-4,4′,6′-Trimethoxychalcone; 3,5-Dinitrocatechol (Or-486); Aztreonam; Piceid; Rifaximin; 10-Hydroxycamptothecin; 5,7-Dihydroxyflavone; ANTIMYCIN A; Xanthoxylin; or a pharmaceutically acceptable salt of any such compound, wherein cellular uptake of proNGF is increased at least about 180% than in the absence of the compound, when measured under same conditions.

In one embodiment, the method includes administering to the mammal an effective amount of any one or any combination of the following promoting compounds: 10-Hydroxycamptothecin; 5,7-Dihydroxyflavone; ANTIMYCIN A; Xanthoxylin; or a pharmaceutically acceptable salt of any such compound, wherein cellular uptake of proNGF is increased at least about 400% than in the absence of the compound, when measured under same conditions.

In another preferred embodiment, the method includes administering to the mammal in need thereof an effective amount of Xanthoxylin,or a pharmaceutically acceptable salt thereof

A “promoting” compound refers to a compound that promotes cellular uptake of proNGF in a cell expressing p75^{NTR} receptor, as described above, i.e., a compound that increases measurable cellular uptake of proNGF by a cell in a given assay in the presence of a compound, relative to the level of measurable cellular uptake in the absence of the compound, when tested under the same conditions.

### Mammal

The present invention provides methods for inhibiting or promoting cellular uptake of proNGF in a mammal in need thereof. A mammal in need of inhibiting cellular uptake of proNGF can include any mammal suffering from a condition that would be alleviated by inhibiting cellular uptake of proNGF. For example, it is known that proNGF binding to p75^{NTR} results in cell death in various neuropathological conditions, such as Alzheimer's disease, spinal cord trauma, axotomy, and retinal dystrophy (Nykjaer et al., 2005).

A mammal in need of promoting cellular uptake of proNGF can include any mammal suffering from a condition that would be alleviated by promoting cellular uptake of proNGF. For example, expression, secretion, and signalling by neurotrophins, proneutrophins, and their receptors, particularly proNGF, NGF, p75^{NTR} and trkA, have been found to cycle in conjunction with human and other mammalian hair follicle cycling, and to be active in skin immune cells, keratinocytes, melanocytes, sebaceous glands, and sweat glands (Adly et al, 2006; Bläsing H et al 2005; Peters et al, 2006a; Peters et al 2006b). Accordingly, promoting cellular uptake of proNGF in a cell expressing p75^{NTR} receptor would be beneficial for conditions relating to human and other mammalian hair follicle cycling, skin immune cells, keratinocytes, melanocytes, sebaceous glands, and sweat glands.

a) Cardiovascular Diseases

In one embodiment, the mammal has suffered from or is suffering from a cardiovascular disease or condition. Cardiovascular disease or condition includes any one of the following: atherosclerosis, acute coronary syndrome, transplantation-induced sclerosis, peripheral limb disease, intermittent claudication, diabetic complication, or thrombosis.

Acute coronary syndrome includes any one of the following: unstable angina, thrombosis, myocardial infarction, plaque rupture, primary restinosis in a coronary artery, primary restinosis in a peripheral artery, secondary restinosis in a coronary artery, or secondary restinosis in a periphery artery.

A diabetic complication includes any one of the following: ischemic heart disease, peripheral artery disease, congestive heart failure, retinopathy, neuropathy, or nephropathy.

Ischemia generally refers to a condition of decreased blood flow to an organ, tissue and/or cell. The decrease in blood flow can be caused by, for example, constriction (e.g., hypoxemic vasoconstriction) or obstruction (e.g., clot, atherosclerotic plaque) of a blood vessel.

Ischemia can occur in any cell, organ, and/or tissue. Examples of cells, organs, and/or tissues which can be subjected to ischemia include neuronal cells (e.g., neurons, ganglia, Schwann cells, astrocytes, oligodendrocytes and microglia), brain, spinal cord, intestinal cells, kidney cells, heart and cardiac muscle cells such as myocytes, etc.

Stroke is a type of cardiovascular disease that generally involves the interruption of blood flow to and/or within the brain. The interruption of blood flow can be due to, for example, a blockage or rupture of an artery or vessel. The blockage typically occurs from a blood clot. As a result of the interruption of blood flow, the brain does not receive sufficient amounts of blood.

b) Microvascular Diseases

The term “microvasculature,” as used herein, refers to the portion of the vascular system composed of small blood vessels, as is known by persons skilled in the art. Examples of small blood vessels include capillaries, precapillary arterioles, post capillary venules, retinal arterioles, glomerular arterioles, cardiac arterioles, the vasa nervorum, and associated capillary beds of the eye, kidney, heart, and central and peripheral nervous system. Such small blood vessels were also described in U.S. Pat. No. 6,951,890.

An abnormality of the microvasculature is any modification of the normal microvasculature. The abnormality is associated with a loss of function of the microvasculature. A loss of function of the microvasculature includes abnormal blood flow, such as excessive blood flow, reduced blood flow, or a complete blockage of blood flow.

A pathological state of a microvasculature includes microvascular disease.

Microvascular diseases include, for example, tremors, ataxias, central nervous system degenerative diseases, neuromuscular degenerative diseases, dyskinesias, encephalitis, and visceral organ microvascular diseases of the heart, pancreas and lung. Such microvasculature diseases are also described in US Published Patent Application 2006-0183733 A1.

Microvascular diseases can affect various organs, such as the kidney, heart, and lungs. A microvascular disease that affects the heart is a cardiovascular pathological state of a microvasculature. Examples of cardiovascular pathological states of a microvasculature include congestive heart failure, peripheral vascular disease, hypertension, myocardial infarction, reperfusion injury, cardiac syndrome X, chronic wound, or diabetic ulcers.

In addition to microvascular diseases, a pathological state of a microvasculature includes microvascular conditions. Microvascular conditions are associated with microvasculature injury or damage. Examples of microvasculature conditions include, for example, retinopathy (the impairment of loss of vision due to blood vessel damage in the eyes); neuropathy (nerve damage and foot problems due to blood vessel damage to the nervous system); and nephropathy (kidney disease due to blood vessel damage in the kidneys). Such microvascular conditions are also described in U.S. Pat. No. 6,262,118.

A pathological state of a microvasculature can be manifested in, for example, altered local blood flow, progressive reversible dilation of small veins, periodic arteriolar vessel constriction, sclerosis of the walls of arterioles, small veins and capillaries, and slowly progressing microcirculatory decompensation. Additional examples of manifestations include a decrease in number of small blood vessels and a frequent thickening of the basement membranes of the attached capillaries and terminal arterioles. Such manifestations are also described in U.S. Pat. No. 4,457,941.

Further manifestations include endothelial cell injury or death and the presence of products of coagulation or thrombosis. Such manifestations are described in U.S. Pat. No. 7,030,083.

A pathological state of a microvasculature can be caused by, for example, diabetes. Additional causes of a pathological state of a microvasculature include a toxin, an immune factor, an infectious agent, a metabolic or physiological stress, or a component of the humoral or cellular immune system, or may be as of yet unidentified. Such causes are described in U.S. Pat. No. 7,030,083.

c) Trauma to the CNS or PNS

In yet another embodiment, the mammal has suffered from or is suffering from trauma to the central nervous system, trauma to the peripheral nervous system, epilepsy-related brain damage, neurotoxic compound poisoning, radiation-induced brain damage, infectious disease of the central nervous system, infectious disease of the peripheral nervous system, stroke, ischemia, or neurodegenerative disease or condition.

Any type of trauma to the nervous system may be treated by the methods of the claimed invention. For example, trauma of the central nervous system (CNS) or peripheral nervous system (PNS) include, but are not limited to, spinal cord injuries, spinal cord lesions, other CNS pathway lesions, as well as injuries to the PNS, such as injuries to a nerve or neuron of the PNS and axon damage resulting in demyelination of the PNS. Such trauma can arise from either physical injury or disease. Any mammal suffering from a trauma of the CNS or PNS may be a mammal in need thereof, in accordance with the methods of the present invention.

For example, spinal cord injury refers to any damage to the spinal cord. The damage typically results in loss of function, such as mobility or feeling. Damage to the spinal cord can occur, for example, as a result or trauma (car accident, gunshot, falls, etc.) or disease (polio, spina bifida, Friedreich's Ataxia, etc).

Any injury to the spinal cord can be treated in accordance with the method of the present invention. For example, the injury can be a complete injury to the spinal cord. Complete injury typically refers to the lack of function (e.g., no sensation and no voluntary movement) below the site of injury. Both sides of the body are usually affected.

Alternatively, the injury may be an incomplete injury to the spinal cord. An incomplete injury generally refers to some function below the site of injury. For instance, a person with an incomplete injury may be able to move one limb more than another, may be able to feel parts of the body that cannot be moved, or may have more functioning on one side of the body than the other, etc.

Injury or trauma may also be caused by epilepsy; infectious disease, such as bacterial or viral meningitis and meningo-encephalitis, or prion diseases; poisonings with neurotoxic compounds; or radiation.

A neurodegenerative disease or condition includes any one of the following: Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Neuroborreliosis, Parkinson disease, Pelizaeus-Merzbacher Disease, Pick's disease, Primary lateral sclerosis, Prion diseases, Refsum's disease, Sandhoff disease, Schilder's disease, Schizophrenia, Spinocerebellar ataxia, Spinal muscular atrophy, Steele-Richardson-Olszewski disease, Tabes dorsalis, depression, psychiatric disorder, or dementia.

As stated above, proNGF induces death of neurons and oligodendrocytes through p75^{NTR}, and its concomitant binding to p75^{NTR} and sortilin has been shown to activate death pathways (Lee et al., 2001; Beattie et al., 2002; Nykjaer et al., 2004; Domeniconi M et al, 2007). For example, proNGF binding to p75^{NTR} can induce apoptosis after injury. Other recent evidence suggests that p75^{NTR} interacting with proteins such as proNGF might mediate cell death in various neuropathological conditions (Nykjaer et al., 2005). Accordingly, inhibiting cellular uptake of proNGF in a cell expressing p75^{NTR} receptor would be beneficial for mammals that have suffered or are suffering from trauma to the CNS or PNS.

In another embodiment, the mammal has suffered from or is suffering from hair loss or hearing loss.

d) Diseases and Conditions of the Skin

Diseases and conditions of the skin amenable to methods to inhibit proNGF uptake include those selected from the following: baldness, lipoma, and melanoma.

Diseases and conditions of the skin amenable to methods to promote proNGF uptake include those selected from the following: unwanted hair, acne, actinic keratosis, atopic dermatitis, chloracne, dermatofibroma, eczema, epidermolysis bullosa, hemangioma, hyperhidrosis, keloid, keratoacanthoma, keratosis pilaris, melasma, perioral dermatitis, photoallergy, photosensitivity, pityriasis rosea, pityriasis rubra pilaris, porphyria, psoriasis, pyoderma, Raynaud's disease, rosacea, scleroderma, sebaceous cyst, seborrheic keratosis, seborrhoeic dermatitis, skin cancer, skin tags, spider veins, squamous cell carcinoma, stasis dermatitis, tungiasis, warts, and xeroderma pigmentosa.

e) Cancers

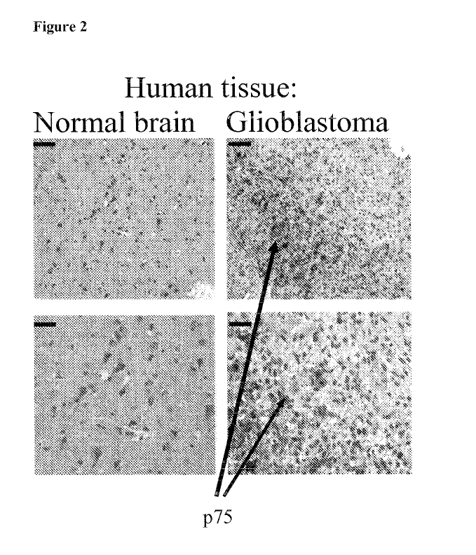

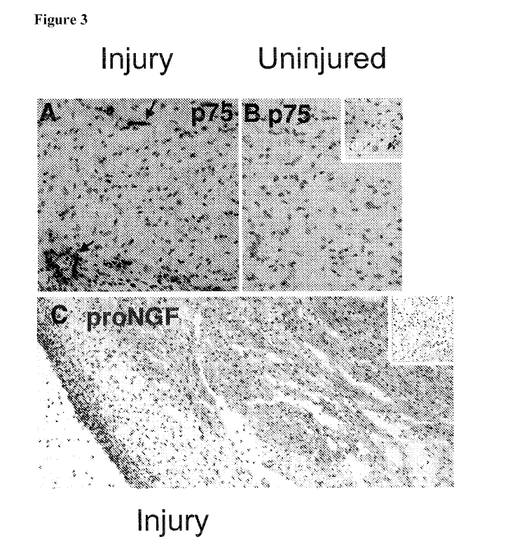

In yet another embodiment, the mammal has suffered from or is suffering from cancer. For example, the p75 receptor is overexpressed in certain cancers, including cancers of neuro-ectodermal origin such as glioblastoma and melanoma. Once these cancer cells spread, they are largely incurable malignancies with few treatment options. P75 is expressed most highly by cells at the invasive edge of these tumors (FIG. 2). ProNGF is chemotactic, inducing the migration and invasion of p75 expressing tumor cells. See for examples Bassili et al., 2009, Chan et al., 2009; Schulte et al., 2009.

### Compounds

Inhibiting compounds useful in the methods of the present invention include 2′,2′-Bisepigallocatechin Monogallate; 2-Aminoethyldiphenyl Boronate; 2-Isopropyl-3-Methoxycinnamic Acid; 4,4′-diisothiocyanostilbene-2,2′-disulfonic acid Sodium Salt; 5-Chloroindole-2-Carboxylic Acid; 5-Fluoroindole-2-Carboxylic Acid; Acetohydroxamic Acid; Acetyl Tyrosine Ethyl Ester; Almotriptan; Almotriptan; Apramycin; Atorvastatin Calcium; Aurintricarboxylic Acid; Aurothioglucose; Butamben; Calcein; Crustecdysone; 2,3,4,6-tetrakis-O-(3-nitropropanoyl)-alpha-L-galactopyranose; Epicatechin Monogallate; Epigallocatechin-3-Monogallate; Glyburide; Hematein; Hinokitiol; Homosalate; Hydroquinone; Indole-2-Carboxylic Acid; Iopanic Acid; Irigenol; iriginol hexaaceatate; Levodopa; Lithium Citrate; Lovastatin; Meclizine Hydrochloride; Miglitol; N-(9-Fluorenylmethoxycarbonyl)-L-Leucine; Ononetin; Oxaprozin; Oxybenzone; Perillic Acid (−); Perindopril Erbumine; Prazosin Hydrochloride; Prenyletin; Quercetin Pentamethyl Ether; Ribostamycin Sulfate; Robustic Acid; Sparteine Sulfate; Sulconazole Nitrate; Sumaresinolic Acid; Suramin; Tannic Acid; Theaflavin; Theaflavin Digallate; or a pharmaceutically acceptable salt of any such compound.

Promoting compounds that are useful in the methods of the present invention include Trichlormethine; 6,7-Dihydroxyflavone; Tangeritin; 3′,4′-Dihydroxyflavone; Amsacrine Hydrochloride; Putrescine Dihydrochloride; Flunixin Meglumine; 3-Benzyloxy-4,4-Bisnor-8,11,13-Podocarpatriene; 2-Acetylpyrrole; Dantron; 2,3,4-Trihydroxy-4′-Ethoxybenzophenone; 8-Cyclopentyltheophylline; Aminocyclopropanecarboxylic Acid; 2′,3-Dihydroxy-4,4′,6′-Trimethoxychalcone; 3,5-Dinitrocatechol (Or-486); Aztreonam; Piceid; Rifaximin; 10-Hydroxycamptothecin; 5,7-Dihydroxyflavone; ANTIMYCIN A; Xanthoxylin; or a pharmaceutically acceptable salt of any such compound.

Such exemplary compounds useful in the methods of the present invention are listed in Table 1 and/or Table 2. The listed compounds are known in the art.

Lithium carbonate is a compound which has been used for decades in humans as a mood stabilizer, predominantly in patients with bipolar disorder. In humans, oral sustained released preparations are given two to three times a day, to achieve serum concentrations of 0.6-0.8 mmol; the major toxicities are reversible renal impairment and anemia (Grandjean et al, 2009). As our studies demonstrate efficacy at 10 micromolar (approximately 1/70 the clinical dose in humans) these toxicities are not anticipated, and indeed prior studies have demonstrated no clinical toxicity when delivered to mice at 1 mEq/kg (73 mg/kg) subcutaneously for 75 days (Fornai et al 2008), a dose that is roughly 50 time higher than we propose. Preliminary dose response studies document that in rats, 100 mg/kg subcutaneously yields a peak serum concentration of 3 mM, and trough of 300 micromolar at 12 hours. Clearance studies in humans and monkeys document that brain concentrations are approximately half of serum concentrations. In one embodiment of the invention, 6 mg/kg is administered subcutaneously every 12 hours to maintain a trough serum concentration of 20 micromolar (anticipating 10 micromolar in the brain). As mentioned above, at much higher doses than those proposed here, and used in ALS models in mice, and in a non-randomized clinical trial of ALS patients lithium promotes survival overall survival (Fornai et al., 2008); also lithium is neuroprotective in kainate toxicity (Busceti et al, 2007).

L-DOPA. The pharmacokinetics of levodopa/benserizide hydorchloride (a peripheral DOPA decarboxylase inhibitor) in mice delivered by intraperitoneal injection, and oral delivery of comparable formulations in human yields plasma levels of levodopa of 1.5 to 6 microM (Physicians Desk reference; Santini et al., 2009a, 2009b; Bergmann et al., 1974). Although there are no available data on CSF levels of L-dopa, the major metabolite, homovanillic acid, is present at 0.8 to 1 micromolar in Parkinsonian patients 8 hours after a single delivery of L-dopa, suggesting that this is a clinically achievable dose (Bergmann et al., 1974). In mice, doses of 5 mg/kg to 20 mg/kg levodopa and a fixed dose of 12 mg/kg benserizide hydrochloride have been given in many studies, although at the higher doses, dyskinesia occurs in most animals after several days. In a preferred embodiment, doses are administered of 5 mg/kg to 20 mg/kg levodopa and a fixed dose of 12 mg/kg benserizide hydrochloride One report suggests that intracellular dopamine levels can be toxic to dopaminergic neurons. It is important to stress that this report employed L-dopa concentrations of 100 to 500 micromolar (100-500 fold higher than the embodiments described herein) were required to observe these effects.

The compounds can be in the form of a pharmaceutically acceptable salt. The term “pharmaceutically acceptable salt” refers to a well-tolerated, nontoxic salt prepared from any basic or acidic compound mentioned above, and an acid or base, respectively. The acids may be inorganic or organic acids of any one of the compounds mentioned above. Examples of inorganic acids include hydrochloric, hydrobromic, nitric hydroiodic, sulfuric, and phosphoric acids. Examples of organic acids include carboxylic and sulfonic acids. The radical of the organic acids may be aliphatic or aromatic. Some examples of organic acids include formic, acetic, phenylacetic, propionic, succinic, glycolic, glucuronic, maleic, furoic, glutamic, benzoic, anthranilic, salicylic, phenylacetic, mandelic, embonic(pamoic), methanesulfonic, ethanesulfonic, panthenoic, benzenesulfonic, stearic, sulfanilic, alginic, tartaric, citric, gluconic, gulonic, arylsulfonic, and galacturonic acids. Appropriate organic bases may be selected, for example, from N,N-dibenzylethylenediamine, chloroprocaine, choline, diethanolamine, ethylenediamine, meglumine(N-methylglucamine), and procaine.

Throughout this specification, parameters are defined by maximum and minimum amounts. Each minimum amount can be combined with each maximum amount to define a range.

### Administration

The compounds are administered to a mammal, preferably a human. The compound is administered to the mammal in an amount effective in achieving its purpose. The effective amount of the compound to be administered can be readily determined by those skilled in the art, for example, during pre-clinical trials and clinical trials by methods familiar to researchers, physicians, and clinicians. Typical daily doses for administration to humans include approximately 1 mg to 1000 mg.

Any method known to those in the art for contacting a cell, organ or tissue with a compound may be employed. Suitable methods include in vitro, ex vivo, or in vivo methods. In vitro methods typically include cultured samples. For example, a cell can be placed in a reservoir (e.g., tissue culture plate), and incubated with a compound under appropriate conditions suitable for inhibiting or promoting cellular uptake of proNGF. Suitable incubation conditions can be readily determined by those skilled in the art.

Ex vivo methods typically include cells, organs or tissues removed from a mammal, such as a human. The cells, organs or tissues can, for example, be incubated with the compound under appropriate conditions. The contacted cells, organs or tissues are normally returned to the donor, placed in a recipient, or stored for future use. Thus, the compound is generally in a pharmaceutically acceptable carrier.

In vivo methods are typically limited to the administration of a compound, such as those described above, to a mammal, preferably a human. The compounds useful in the methods of the present invention are administered to a mammal in an amount effective in inhibiting or promoting cellular uptake of proNGF. The effective amount is determined during pre-clinical trials and clinical trials by methods familiar to physicians and clinicians.

An effective amount of a compound useful in the methods of the present invention, preferably in a pharmaceutical composition, may be administered to a mammal in need thereof by any of a number of well-known methods for administering pharmaceutical compounds. The compound may be administered systemically or locally.

For example, the compound may be administered orally, intravenously, intranasally, intramuscularly, subcutaneously, or transdermally. Other routes of administration include intracerebroventricularly or intrathecally. Intracerebroventiculatly refers to administration into the ventricular system of the brain. Intrathecally refers to administration into the space under the arachnoid membrane of the spinal cord. Thus intracerebroventricular or intrathecal administration may be preferred for those diseases and conditions which affect the organs or tissues of the central nervous system.

The compounds useful in the methods of the invention may also be administered to mammals by sustained release, as is known in the art. Sustained release administration is a method of drug delivery to achieve a certain level of the drug over a particular period of time. The level typically is measured by serum or plasma concentration.

A description of methods for delivering a compound by controlled release can be found in international PCT Application No. WO 02/083106. The PCT application is incorporated herein by reference in its entirety. Other controlled release agents are described, for example, in U.S. Pat. Nos. 5,567,439; 6,838,094; 6,863,902; and 6,905,708. The controlled release agents and methods for making them in these patents are incorporated herein by reference.

Any formulation known in the art of pharmacy is suitable for administration of the compounds useful in the methods of the present invention. For oral administration, liquid or solid formulations may be used. Some examples of formulations include tablets, gelatin capsules, pills, troches, elixirs, suspensions, syrups, wafers, chewing gum and the like. The compounds can be mixed with a suitable pharmaceutical carrier (vehicle) or excipient as understood by practitioners in the art. Examples of carriers and excipients include starch, milk, sugar, certain types of clay, gelatin, lactic acid, stearic acid or salts thereof, including magnesium or calcium stearate, talc, vegetable fats or oils, gums and glycols.

For systemic, intracerebroventricular, intrathecal, topical, intranasal, subcutaneous, or transdermal administration, formulations of the compounds useful in the methods of the present inventions may utilize conventional diluents, carriers, or excipients etc., such as those known in the art to deliver the compounds. For example, the formulations may comprise one or more of the following: a stabilizer, a surfactant, preferably a nonionic surfactant, and optionally a salt and/or a buffering agent. The compound may be delivered in the form of an aqueous solution, or in a lyophilized form.

The stabilizer may, for example, be an amino acid, such as for instance, glycine; or an oligosaccharide, such as for example, sucrose, tetralose, lactose or a dextran. Alternatively, the stabilizer may be a sugar alcohol, such as for instance, mannitol; or a combination thereof Preferably the stabilizer or combination of stabilizers constitutes from about 0.1% to about 10% weight for weight of the compound. The surfactant is preferably a nonionic surfactant, such as a polysorbate. Some examples of suitable surfactants include Tween20, Tween80; a polyethylene glycol or a polyoxyethylene polyoxypropylene glycol, such as Pluronic F-68 at from about 0.001% (w/v) to about 10% (w/v).

The salt or buffering agent may be any salt or buffering agent, such as for example, sodium chloride, or sodium/potassium phosphate, respectively. Preferably, the buffering agent maintains the pH of the pharmaceutical composition in the range of about 5.5 to about 7.5. The salt and/or buffering agent is also useful to maintain the osmolality at a level suitable for administration to a human or an animal Preferably the salt or buffering agent is present at a roughly isotonic concentration of about 150 mM to about 300 mM.

The formulations of the compounds useful in the methods of the present invention may additionally contain one or more conventional additive. Some examples of such additives include a solubilizer such as, for example, glycerol; an antioxidant such as for example, benzalkonium chloride (a mixture of quaternary ammonium compounds, known as “quats”), benzyl alcohol, chloretone or chlorobutanol; anaesthetic agent such as for example a morphine derivative; or an isotonic agent etc., such as described above. As a further precaution against oxidation or other spoilage, the pharmaceutical compositions may be stored under nitrogen gas in vials sealed with impermeable stoppers.

### EXAMPLES

### Example 1

Compounds from the SpecPlus collection (MicroSource Discovery Systems, Groton, Conn.) were assayed in cells that express p75^{NTR} and sortilin. The compounds in the collection are primarily Food and Drug Administration (FDA)-approved compounds or natural products.

### Example 2

Lithium citrate, L-dopa, and lovastatin have excellent toxicity profiles in human, and clinically achievable doses can be obtained. In addition, these three compounds have been further evaluated in proNGF-induced SCG neuronal cell death. Each exhibits dose dependent inhibition of apoptosis, without toxicity, and >65% reduction in apoptosis is demonstrated upon co-incubation with 1 micromolar concentrations of each compound.

### Example 3

### ProNGF is Induced, Secreted and Plays a Critical Role in Neuronal Death After Seizures.

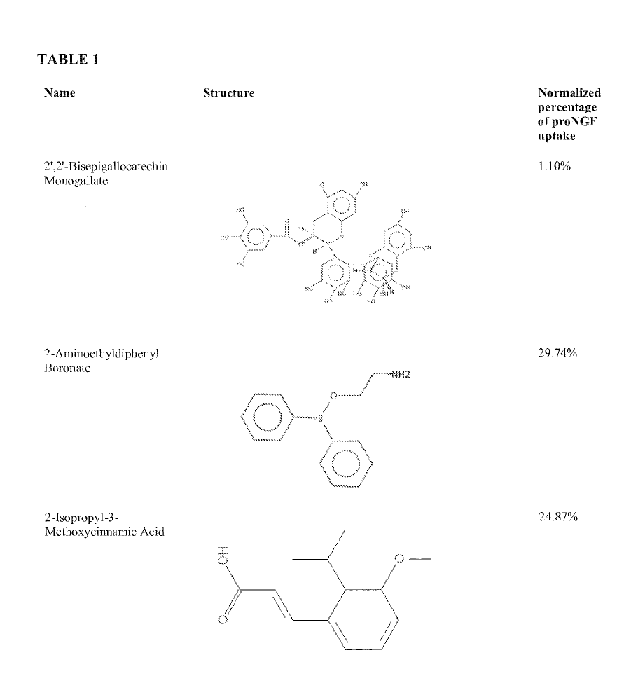

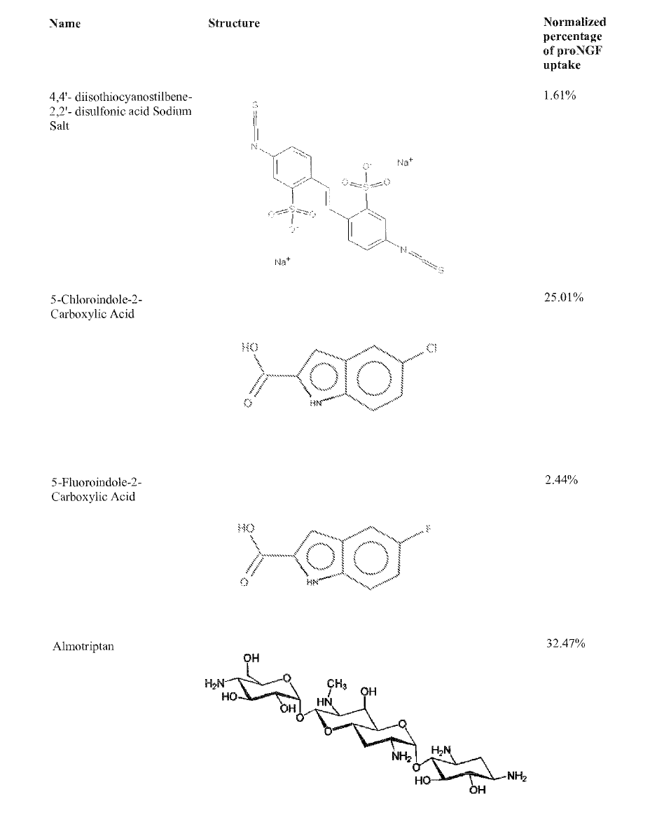

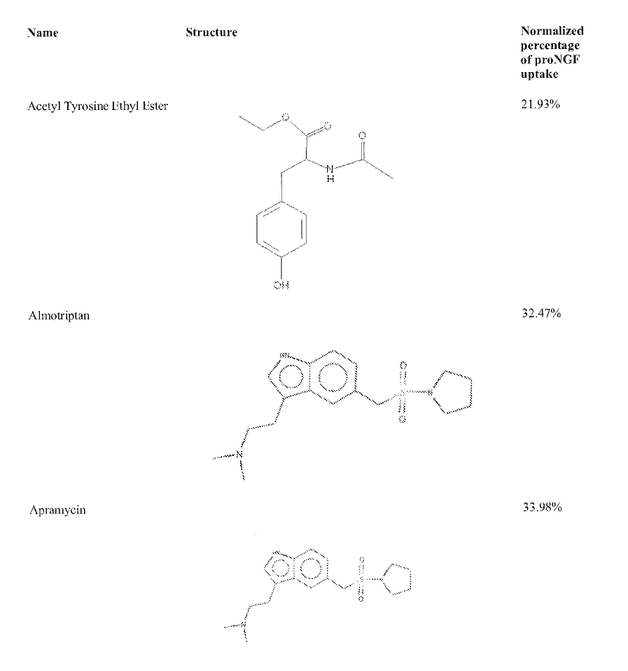

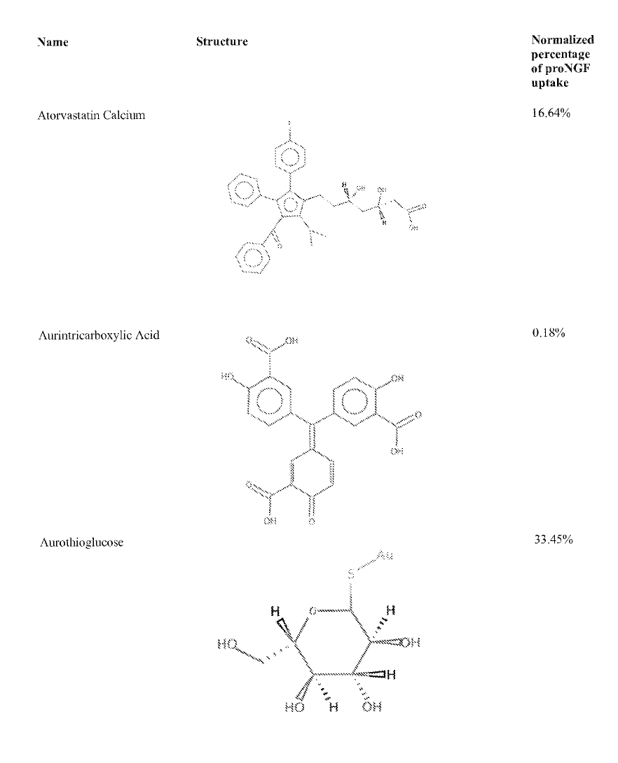

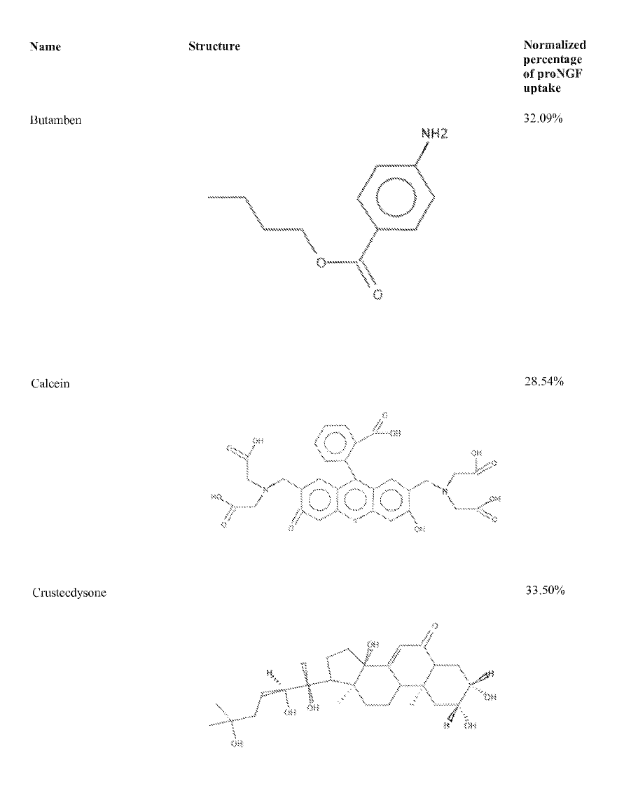

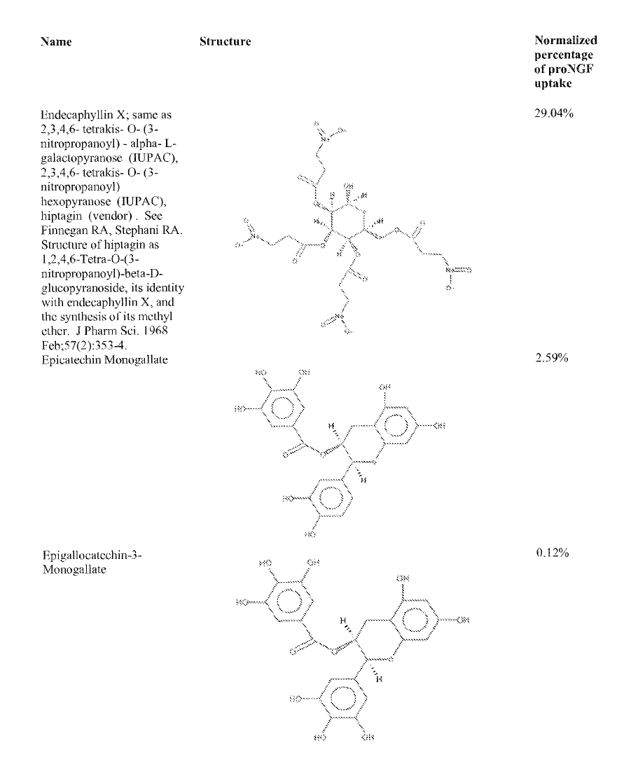

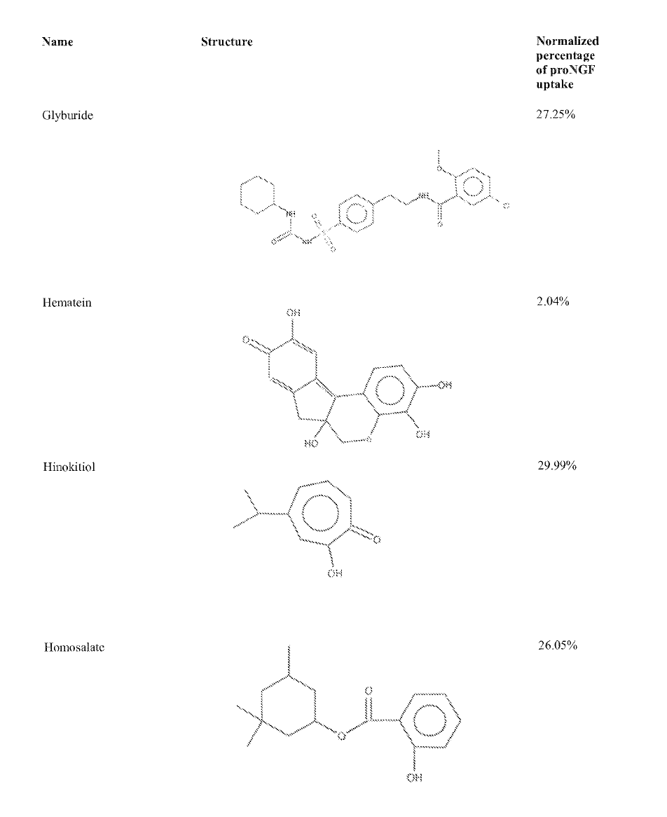

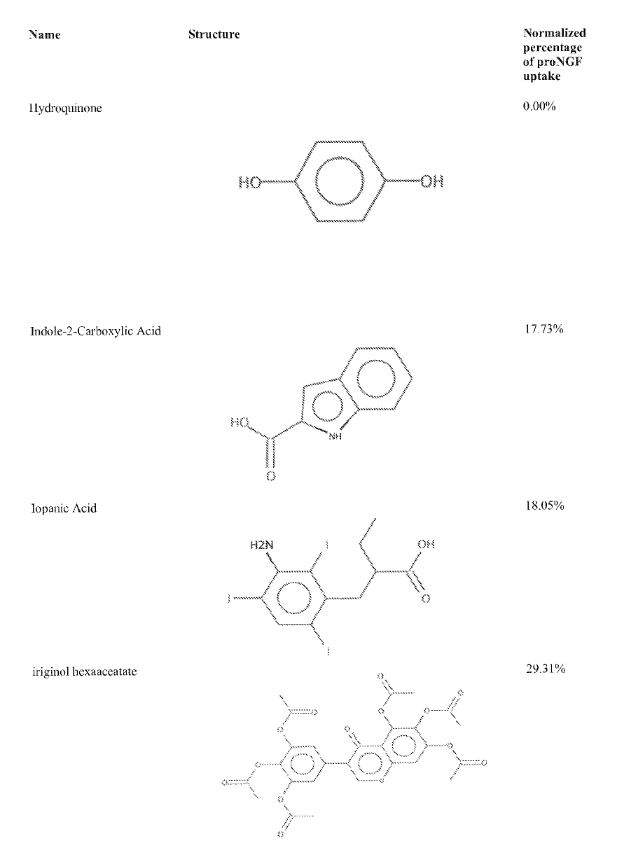

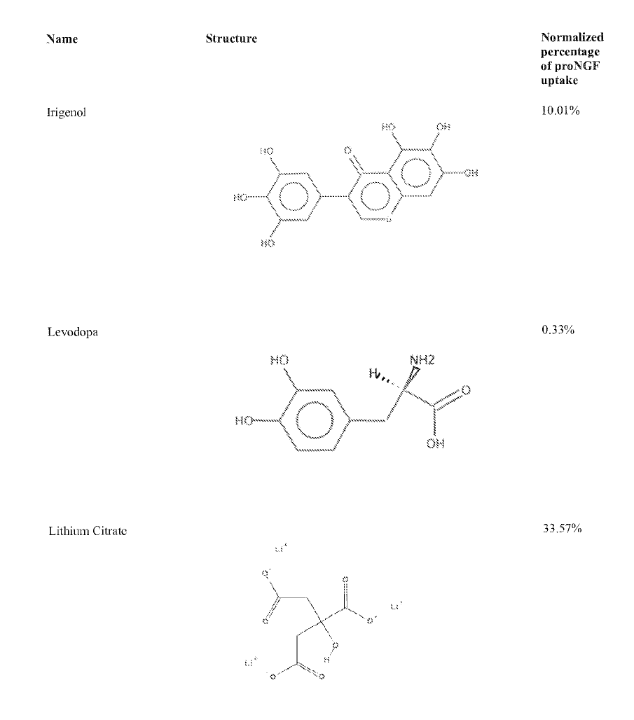

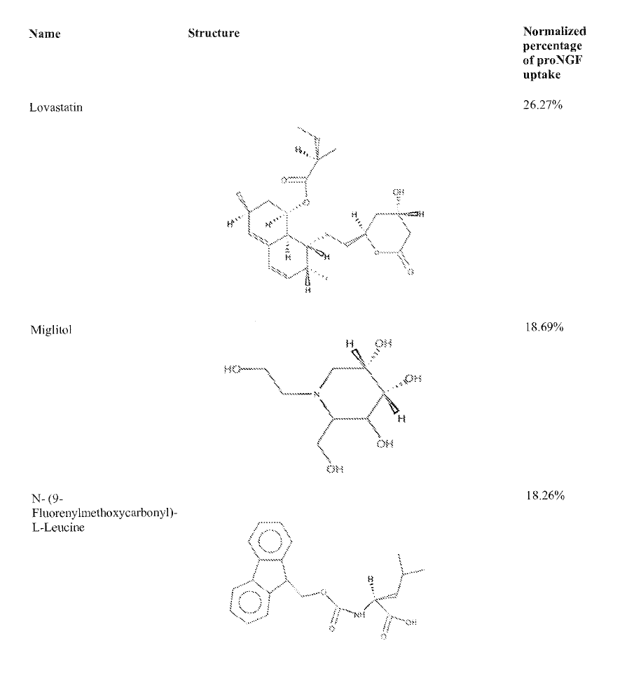

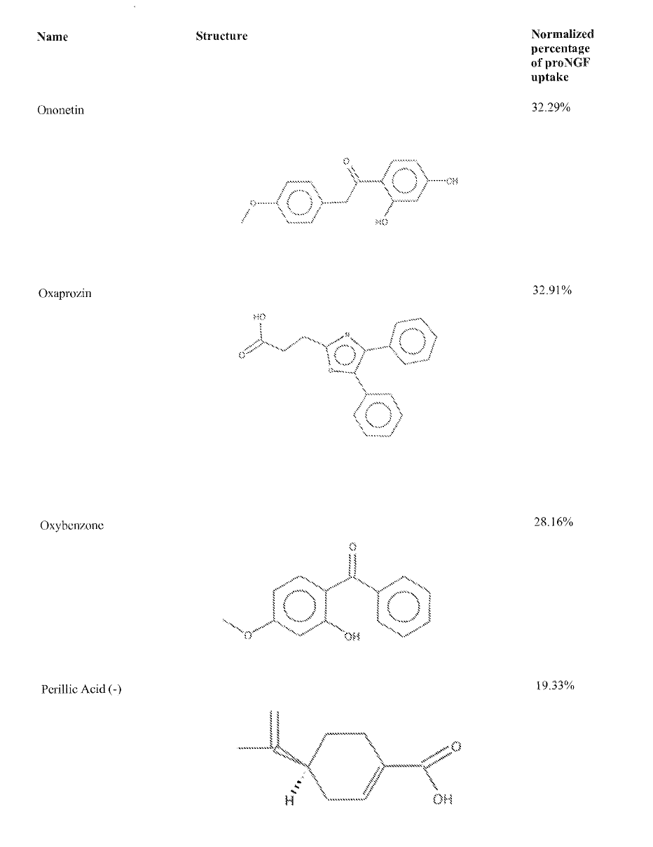

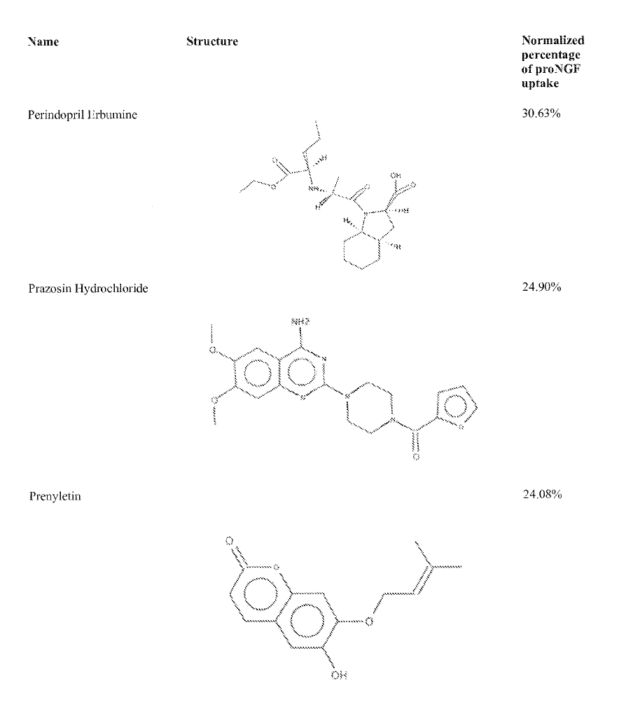

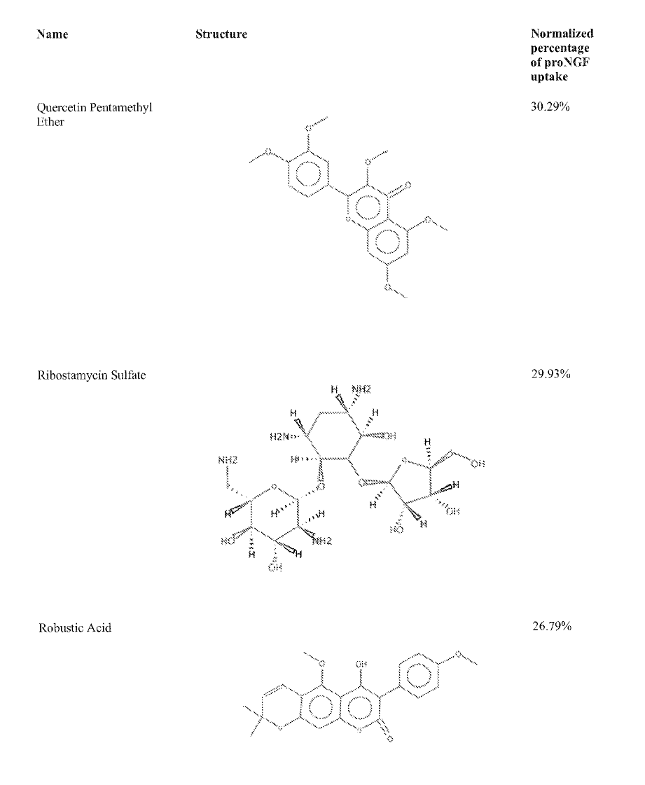

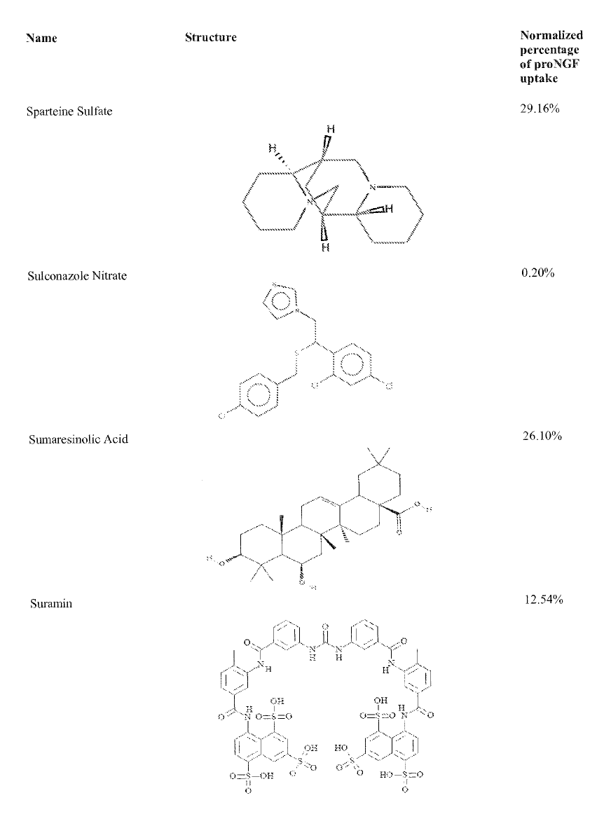

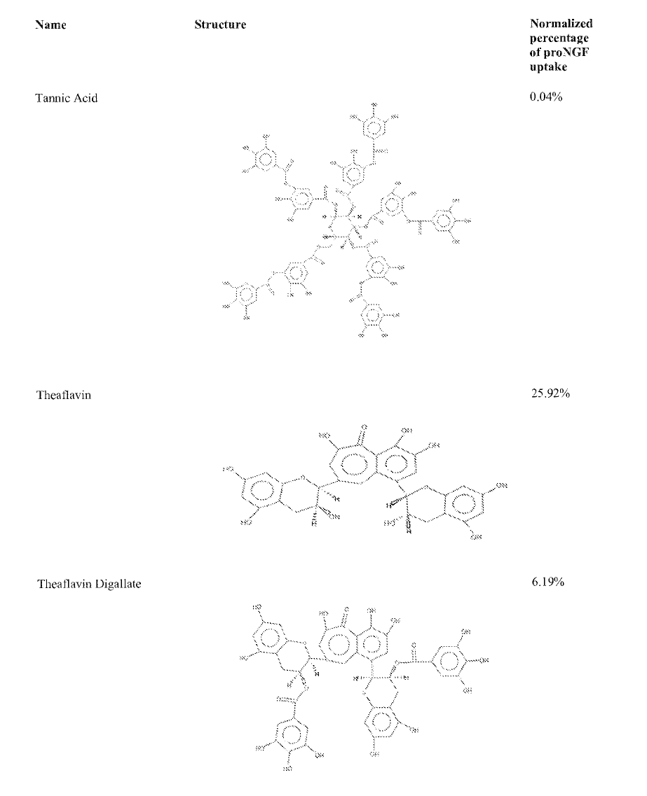

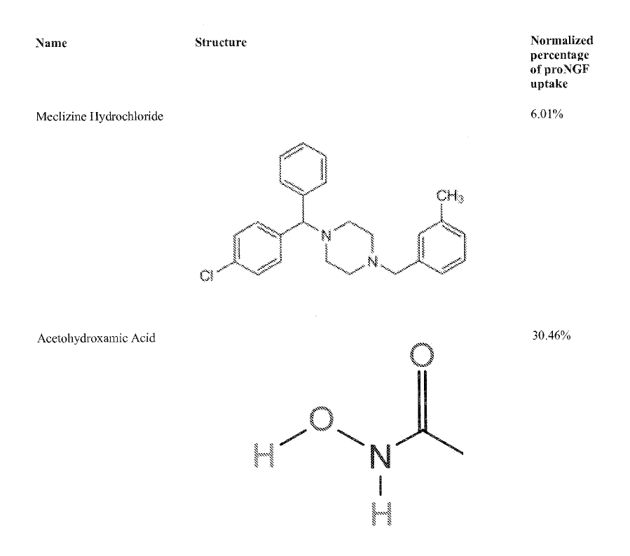

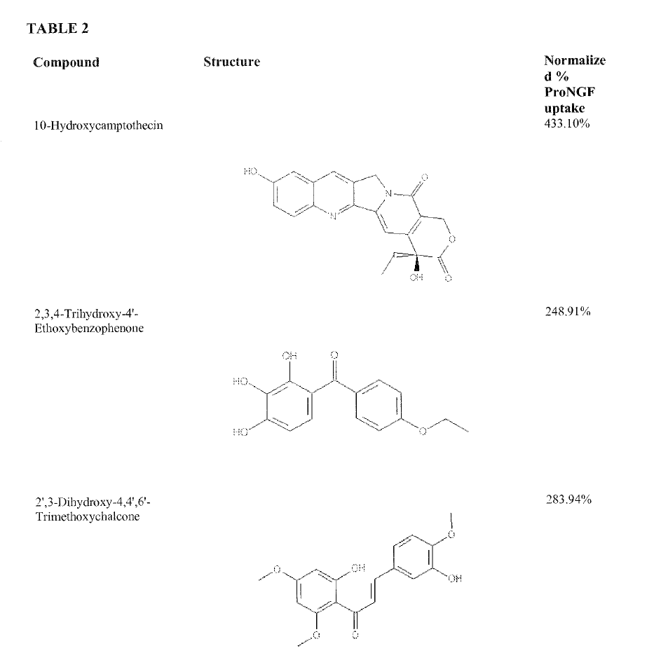

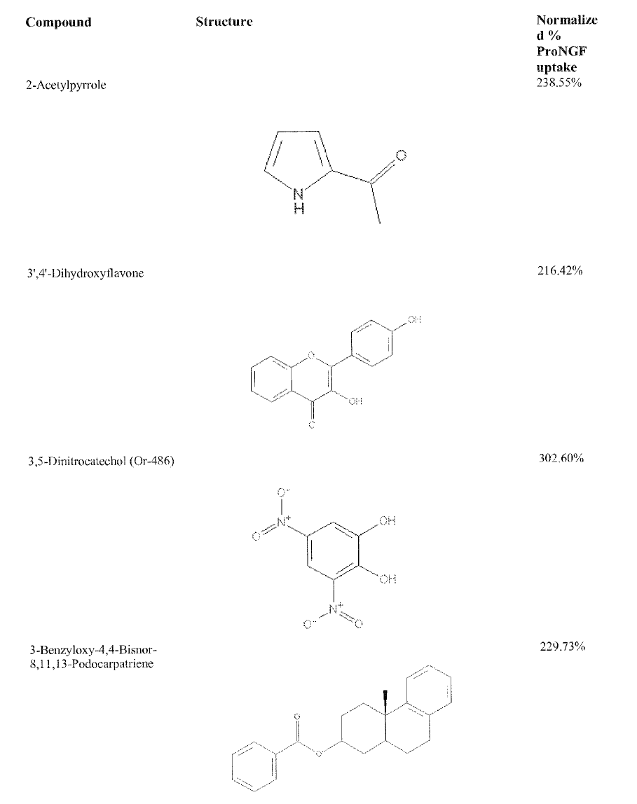

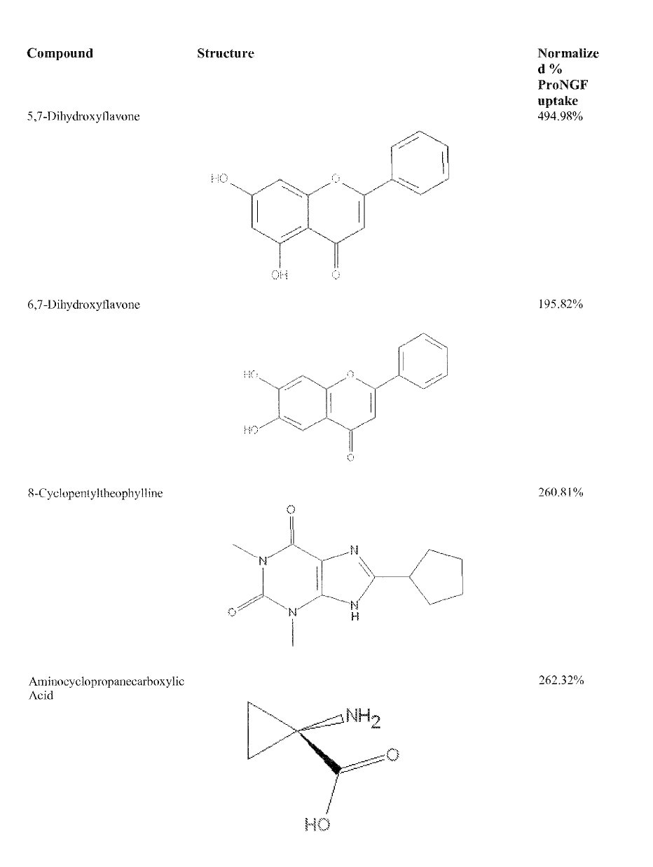

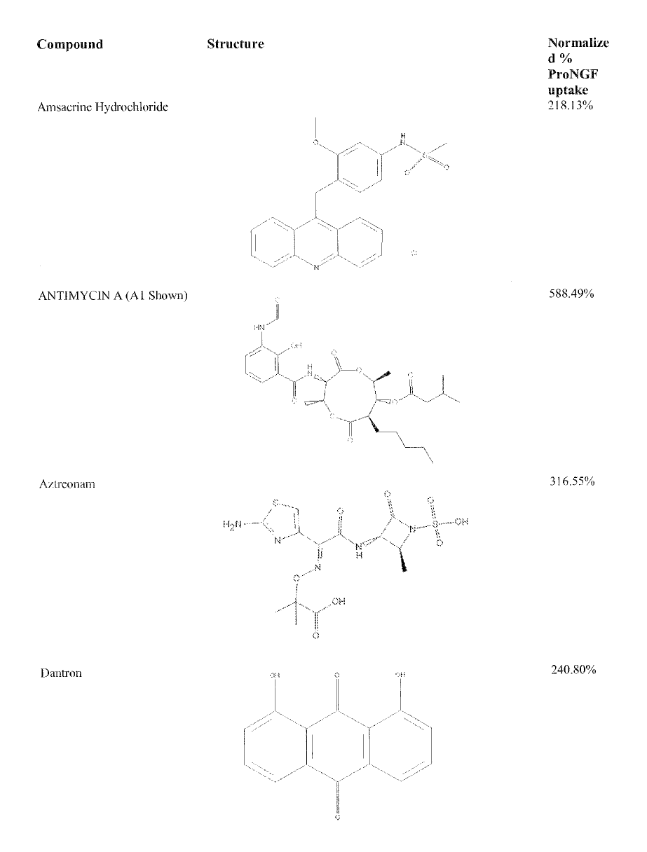

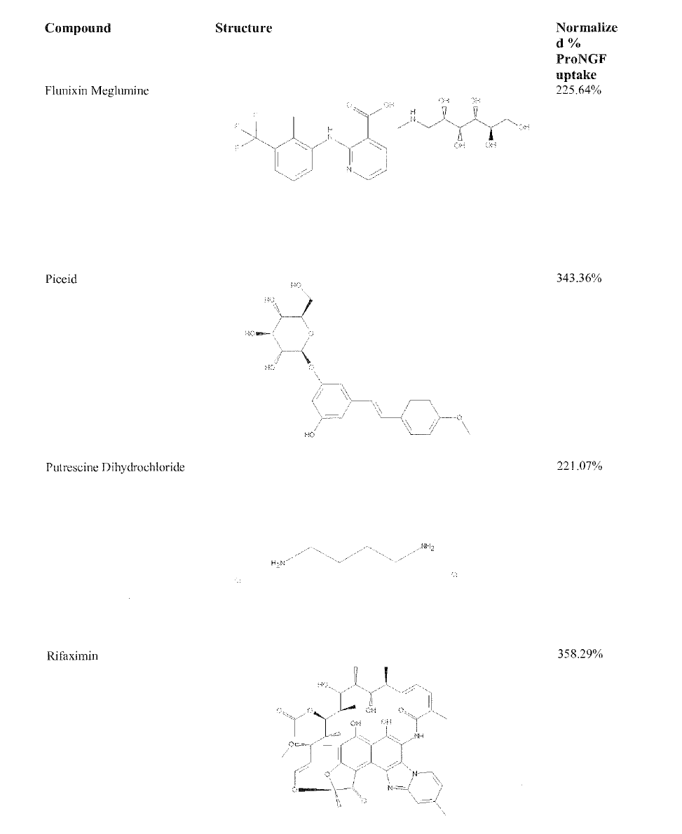

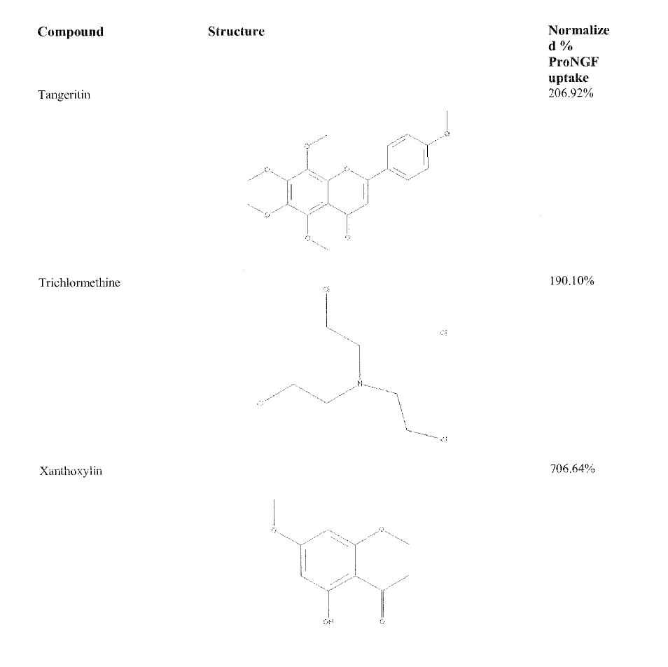

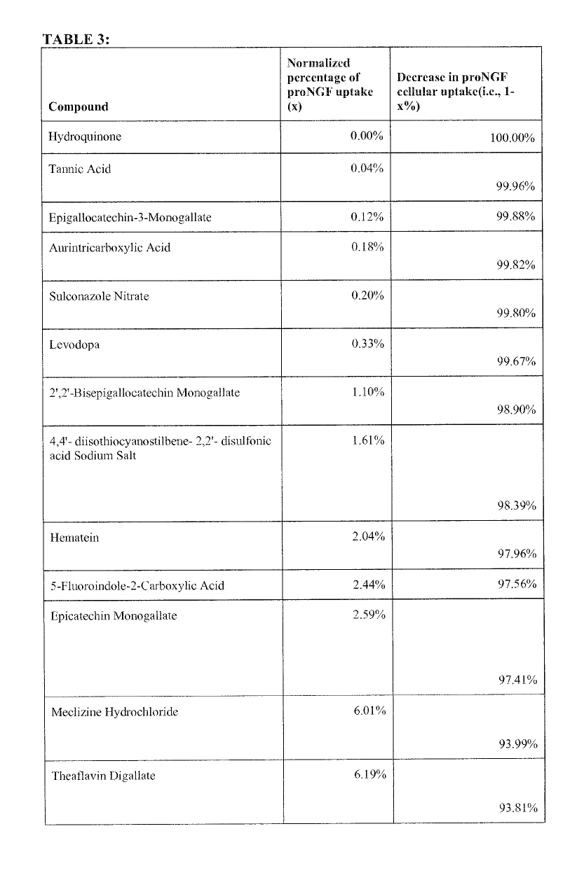

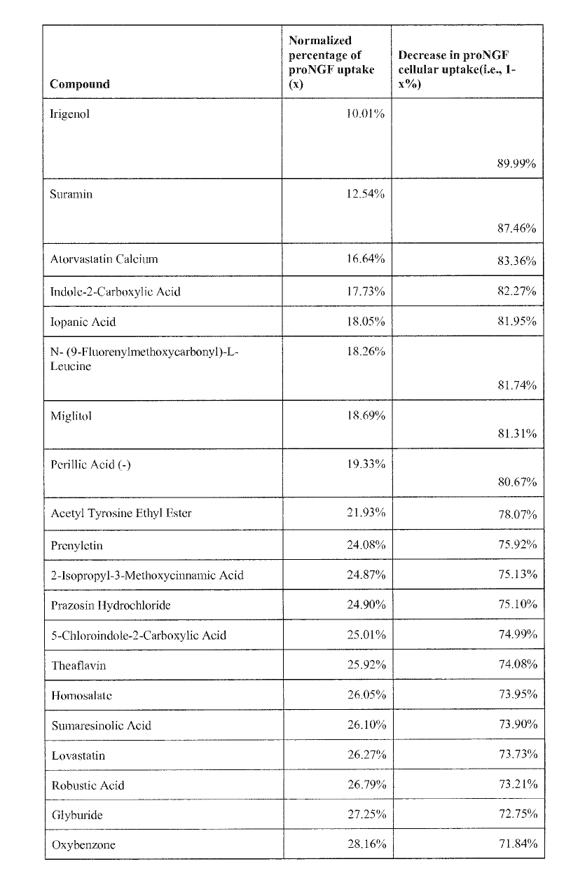

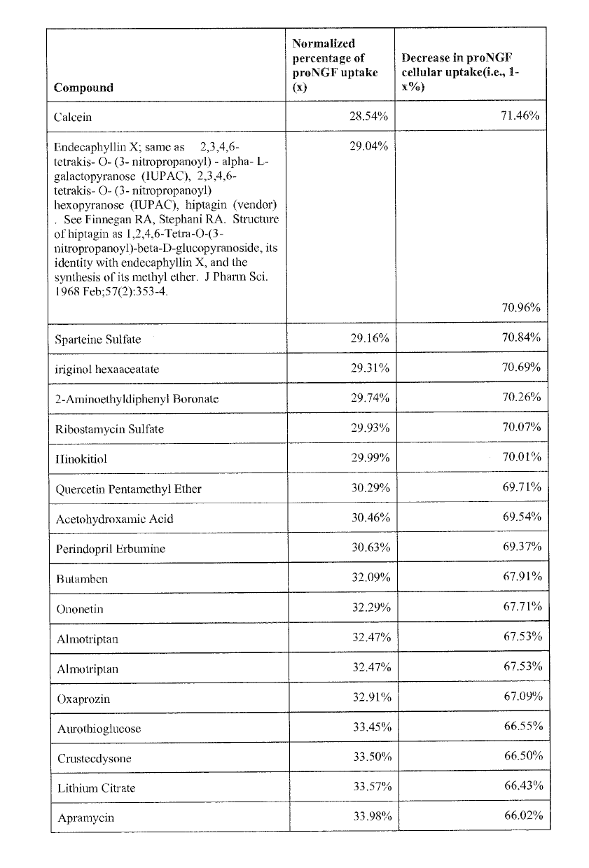

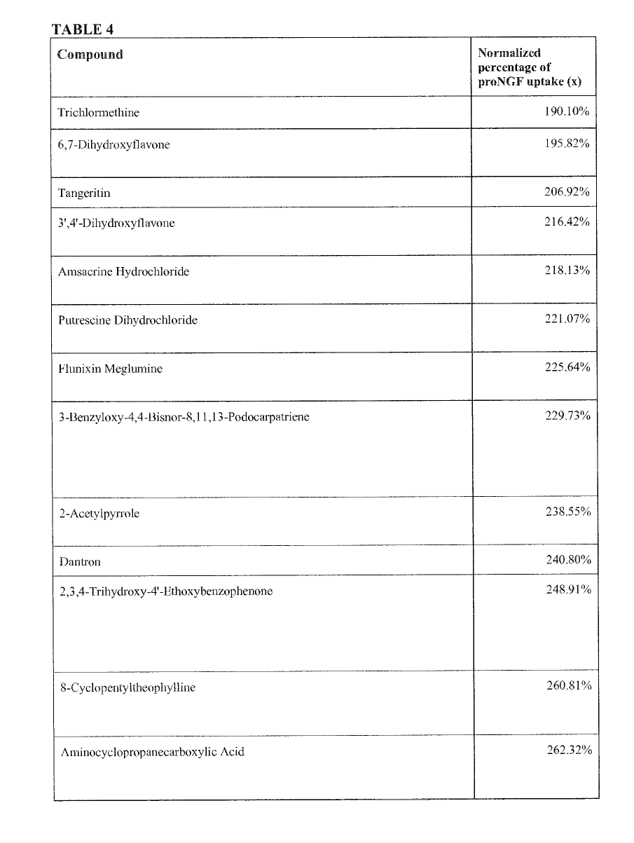

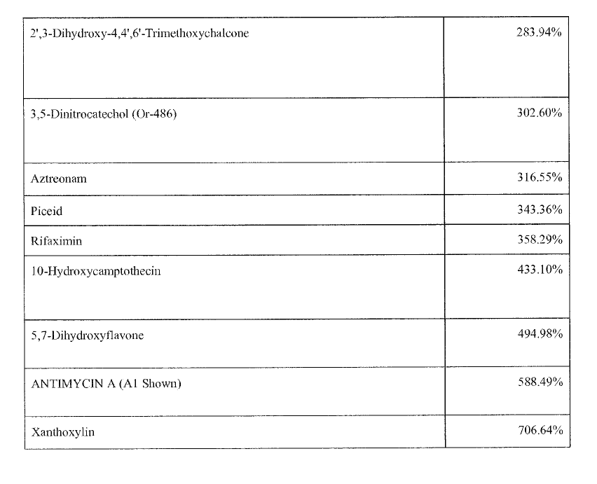

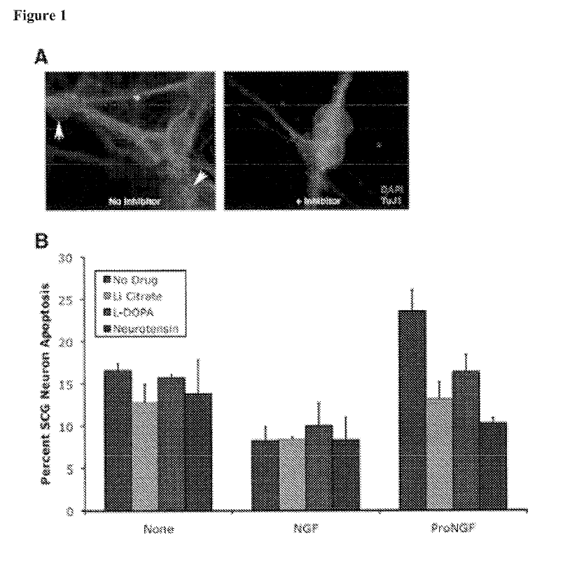

A recent collaborative study assessed the expression and biological actions of proNGF following seizure induced injury to central neurons. Cultured rat SCG neurons (7 DIV) were washed free of NGF and were treated or not with 10 ng/ml NGF or 5 ng/ml proNGF in the presence or absence of 10 μM Lithium Citrate or L-DOPA, or 20 μM neurotensin. Results are shown in FIG. 1. (A) Neuronal apoptosis was assessed morphologically by DAPI and TuJ1 double staining and (B) quantitated as percentage of SCG neuron apoptosis. The numbers represent results from three independently conducted experiments. Error bars represent S.E.M.

These studies suggest that proNGF is a locally induced cytokine that is synthesized in response to injury, and mediates neuronal death in vivo.

### REFERENCES

Adly M A, Assaf H A, Nada E A, Soliman M, Hussein M. (2006) Expression of nerve growth factor and its high-affinity receptor, tyrosine kinase A proteins, in the human scalp skin. J Cutan Pathol. 33(8):559-68.
Bassili M, Birman E, Schor N F, Saragovi H U. (2009) Differential roles of Trk and p75 neurotrophin receptors in tumorigenesis and chemoresistance ex vivo and in vivo. Cancer Chemother Pharmacol.
Beattie M S, Harrington A W, Lee R, Kim J Y, Boyce S L, Longo F M, Bresnahan J C, Hempstead B L, Yoon S O (2002) ProNGF induces p75-mediated death of oligodendrocytes following spinal cord injury. Neuron 36:375-386.
Bergmann S, Curzon G, Friedel F, Godwin-Austen R B, Marsden C D, Parkes J D. (1974) The absorption and metabolism of a standard oral dose of levodopa in patients with parkinsonism. Br J Clin Pharmacol. 1: 417-424.
Bläsing H, Hendrix S, Paus R. (2005) Pro-inflammatory cytokines upregulate the skin immunoreactivity for NGF, NT-3, NT-4 and their receptor, p75^{NTR} in vivo: a preliminary report. Arch Dermatol Res. 296:580-584.
Botchkarev V A, Botchkareva N V, Peters E M, Paus R. (2004) Epithelial growth control by neurotrophins: leads and lessons from the hair follicle. Prog Brain Res. 493-513.
Busceti C L, Biagioni F, Aronica E, Riozzi B, Storto M, Battglia G, Giorno D S Girandini R, Fornai F, Carsacole A (2007) Induction of the Wnt inhibitor, Dickkopf-1, is associated with neurodegeneration related to temporal lobe epilepsy. Epilepsia 48: 694-705.
Caporali A, Pani E, Horrevoets A J, Kraenkel N, Oikawa A, Sala-Newby G B, Meloni M, Cristofaro B, Graiani G, Leroyer A S, Boulanger C M, Spinetti G, Yoon S O, Madeddu P, Emanueli C. (2008) Neurotrophin p75 receptor (p75^{NTR}) promotes endothelial cell apoptosis and inhibits angiogenesis: implications for diabetes-induced impaired neovascularization in ischemic limb muscles. Circ Res. 103:e15-26.
Carter B D, Kaltschmidt C, Kaltschmidt B, Offenhauser N, Bohm-Matthaei R, Baeuerle P A, Barde Y A (1996) Selective activation of NF-kappa B by nerve growth factor through the neurotrophin receptor p75. Science 272:542-545.
Chan M M, Tahan S R. (2009) Low-affinity nerve growth factor receptor (P75 NGFR) as a marker of perineural invasion in malignant melanomas. J Cutan Pathol. 2009 Jul. 13. ahead of print]
Chao M V (2003) Neurotrophins and their receptors: a convergence point for many signalling pathways. Nat Rev Neurosci 4:299-309.
DeFreitas M F, McQuillen P S, Shatz C J (2001) A novel p75^{NTR} signaling pathway promotes survival, not death, of immunopurified neocortical subplate neurons. J Neurosci 21:5121-5129.
Domeniconi M, Hempstead B L, Chao M V. (2007) Pro-NGF secreted by astrocytes promotes motor neuron cell death. Mol Cell Neurosci. 34:271-279.
Donovan M J, Lin M I, Wiegn P, Ringstedt T, Kraemer R, Hahn R, Wang S, Ibañez C F, Rafii S, Hempstead B L. (2000) Brain derived neurotrophic factor is an endothelial cell survival factor required for intramyocardial vessel stabilization. Development 127:4531-4540.

Fahnestock M, Michalski B, Xu B, Coughlin M D (2001) The precursor pro-nerve growth factor is the predominant form of nerve growth factor in brain and is increased in Alzheimer's disease. Mol Cell Neurosci 18:210-220.
Forni F, Longone P, Cafaro L, Kastsiuchenka O, Ferrucci M, Manca M L, Lazzeri G, Spalloni A, Bellio N, Lenzi P, Modugno N, Siciliano G, Isidoro C, Murri L, Rugieri S, Paparelli A. (2008) Lithium delays progression of amyotrophic lateral sclerosis PNAS USA 105: 2052-2057
Grandjean E M, Aubry J M. (2009) Lithium: updated human knowledge using an evidence-based approach: part III: clinical safety. CNS Drugs. 23(5):397-418. Review.
Harrington A, Leiner B, Blechschmitt C, Arevalo J, Lee R, Morl K, Meyer M, Hempstead B, Yoon S, Giehl K (2004) Secreted proNGF is a pathophysiological death-inducing ligand after adult CNS injury. Proc Natl Acad Sci USA 101:6226-6230.
He X L, Garcia K C (2004) Structure of nerve growth factor complexed with the shared neurotrophin receptor p75. Science 304:870-875.
Huang E J, Reichardt L F (2003) Trk receptors: roles in neuronal signal transduction. Annu Rev Biochem 72:609-642.
Kermani P, Rafii D, Jin D K, Whitlock P, Schaffer W, Chiang A, Vincent L, Friedrich M, Shido K, Hackett N R, Crystal R G, Rafii S, Hempstead B L. (2005) Neurotrophins promote revascularization by local recruitment of TrkB+ endothelial cells and systemic mobilization of hematopoietic progenitors. J Clin Invest. 115:653-663.
Lee R, Kermani P, Teng K K, Hempstead B L (2001) Regulation of cell survival by secreted proneurotrophins. Science 294:1945-1948.
Lin M I, Das I, Schwartz G M, Tsoulfas P, Mikawa T, Hempstead B L. (2000) Trk C receptor signaling regulates cardiac myocyte proliferation during early heart development in vivo. Dev Biol. 26:180-191.
Longo F M, Massa S M. (2008) Small molecule modulation of p75 neurotrophin receptor functions. CNS Neurol Disord Drug Targets. 7:63-70. Review.
Longo F M, Manthorpe M, Xie Y M, Varon S (1997) Synthetic NGF peptide derivatives prevent neuronal death via a p75 receptor-dependent mechanism. J Neurosci Res 48:1-17.
Lu B, Pang P T, Woo N H (2005) The yin and yang of neurotrophin action. Nat Rev Neurosci 6:603-614.
Maliartchouk S, Debeir T, Beglova N, Cuello A C, Gehring K, Saragovi H U (2000b) Genuine monovalent ligands of TrkA nerve growth factor receptors reveal a novel pharmacological mechanism of action. J Biol Chem 275:9946-9956.
Massa S M, Xie Y, Yang T, Harrington A W, Kim M L, Yoon S O, Kraemer R, Moore L A, Hempstead B L, Longo F M. (2006) Small, nonpeptide p75^{NTR} ligands induce survival signaling and inhibit proNGF-induced death. J Neurosci. 26:5288-5300.
Mamidipudi V, Li X, Wooten M W (2002) Identification of interleukin 1 receptor-associated kinase as a conserved component in the p75-neurotrophin receptor activation of nuclear factor-kappa B. J Biol Chem 277:28010-28018.
Morrisett R A, Jope R S, Snead O C 3rd. (1987a) Status epilepticus is produced by administration of cholinergic agonists to lithium-treated rats: comparison with kainic acid. Exp Neurol. 98:594-605.
Morrisett R A, Jope R S, Snead O C 3rd. (1987b) Effects of drugs on the initiation and maintenance of status epilepticus induced by administration of pilocarpine to lithium-pretreated rats. Exp Neurol. 97:193-200.

Nykjaer A, Lee R, Teng K K, Jansen P, Madsen P, Nielsen M S, Jacobsen C, Kliemannel M, Schwarz E, Willnow T E, Hempstead B L, Petersen C M (2004) Sortilin is essential for proNGF-induced neuronal cell death. Nature 427:843-848.
Nykjaer A, Willnow T E, Petersen C M (2005) p75NTR-live or let die. Curr Opin Neurobiol 15:49-57.
Pang P T, Teng H K, Zaitsev E, Woo N T, Sakata K, Zhen S, Teng K K, Yung W H, Hempstead B L, Lu B (2004) Cleavage of proBDNF by tPA/plasmin is essential for long-term hippocampal plasticity. Science 306: 487-491
Pedraza C E, Podlesniy P, Vidal N, Arévalo J C, Lee R, Hempstead B, Ferrer I, Iglesias M, Espinet C (2005) Pro-NGF isolated from the human brain affected by Alzheimer's disease induces neuronal apoptosis mediated by p75^{NTR}. Am J Pathol 166:533-543.
Peng S, Wuu J, Mufson E J, Fahnestock M (2004) Increased proNGF levels in subjects with mild cognitive impairment and mild Alzheimer disease. J Neuropathol Exp Neurol 63:641-649.
Peters E M, Hendrix S, Golz G, Klapp B F, Arck P C, Paus R. (2006b) Nerve growth factor and its precursor differentially regulate hair cycle progression in mice. J Histochem Cytochem. 54:275-288.
Peters E M, Stieglitz M G, Liezman C, Overall R W, Nakamura M, Hagen E, Klapp B F, Arck P, Paus R. (2006a) p75 Neurotrophin Receptor-Mediated Signaling Promotes Human Hair Follicle Regression (Catagen). Am J Pathol. 168:221-234.
Reddypalli S, Roll K, Lee H K, Lundell M, Barea-Rodriguez E, Wheeler E F (2005) p75^{NTR}-mediated signaling promotes the survival of myoblasts and influences muscle strength. J Cell Physiol 204:819-829.
Roux P P, Bhakar A L, Kennedy T E, Barker P A (2001) The p75 neurotrophin receptor activates Akt (protein kinase B) through a phosphatidylinositol 3-kinase-dependent pathway. J Biol Chem 276:23097-23104.
Santini E, Heiman M, Greengard P, Valjent E, Fisone G. (2009a) Inhibition of mTOR signaling in Parkinson's disease prevents L-DOPA-induced dyskinesia. Sci Signal 2:ra36.
Santini E, Alcacer C, Cacciatore S, Heiman M, Hervé D, Greengard P, Girault J A, Valjent E, Fisone G. (2009b) L-DOPA activates ERK signaling and phosphorylates histone H3 in the striatonigral medium spiny neurons of hemiparkinsonian mice. J Neurochem 108:621-633
Sariola H. (2001) The neurotrophic factors in non-neuronal tissues. Cell Mol Life Sci. 58:1061-1066. Review.
Sato T, Doi K, Taniguchi M, Yamashita T, Kubo T, Tohyama M. (2006) Progressive hearing loss in mice carrying a mutation in the p75 gene. Brain Res. 1091:224-234.
Salehi A H, Roux P P, Kubu C J, Zeindler C, Bhakar A, Tannis L L, Verdi J M, Barker P A (2000) NRAGE, a novel MAGE protein, interacts with the p75 neurotrophin receptor and facilitates nerve growth factor-dependent apoptosis. Neuron 27:279-288.
Schulte J H, Pentek F, Hartmann W, Schramm A, Friedrichs N, Øra I, Koster J, Versteeg R, Kirfel J, Buettner R, Eggert A. (2009) The low-affinity neurotrophin receptor, p75, is upregulated in ganglioneuroblastoma/ganglioneuroma and reduces tumorigenicity of neuroblastoma cells in vivo. Int J Cancer. 124:2488-2494.
Shmelkov S V, Meeus S, Moussazadeh N, Kermani P, Rashbaum W K, Rabbany S Y, Hanson M A, Lane W J, St Clair R, Walsh K A, Dias S, Jacobson J T, Hempstead B L, Edelberg J M, Rafii S. (2005) Cytokine preconditioning promotes codifferentiation of human fetal liver CD133+ stem cells into angiomyogenic tissue. Circulation 111:1175-1183.
Teng K K, Hempstead B L (2004) Neurotrophins and their receptors: signaling trios in complex biological systems. Cell Mol Life Sci 61:35-48.
Teng H K, Teng K K, Lee R, Wright S, Tevar S, Almeida R D, Kermani P, Torkin R, Chen Z Y, Lee F S, Kraemer R T, Nykjaer A, Hempstead B L (2005) ProBDNF induces neuronal apoptosis via activation of a receptor complex of p75^{NTR} and sortilin. J Neurosci 25:5455-5463.
Volosin M, Song W, Almeida R D, Kaplan D R, Hempstead B L, Friedman W J (2006) Interaction of survival and death signaling in basal forebrain neurons: roles of neurotrophins and proneurotrophins. J Neurosci 26:7756-7766.
Volosin M, Trotter C, Cragnolini A, Kenchappa R S, Light M, Hempstead B L, Carter B D, Friedman W J. (2008) Induction of proneurotrophins and activation of p75NTR-mediated apoptosis via neurotrophin receptor-interacting factor in hippocampal neurons after seizures. J Neurosci. 28:9870-9879.
Wang J J, Rabizadeh S, Tasinato A, Sperandio S, Ye X, Green M, Assa-Munt N, Spencer D, Bredesen D E (2000) Dimerization-dependent block of the proapoptotic effect of p75^{NTR}. J Neurosci Res 60:587-593.
Wang S, Bray P, McCaffrey T, March K, Hempstead B L, Kraemer R. (2000) p75(NTR) mediates neurotrophin-induced apoptosis of vascular smooth muscle cells. Am J Pathol. 157:1247-1258.

What is claimed is:

1. A method of inhibiting cellular uptake of pro-nerve growth factor (proNGF) in a cell expressing neurotrophin p75 receptor in a mammal in need thereof, the method comprising administering to the mammal an effective amount of any one or any combination of the following inhibiting compounds: 2′,2′-Bisepigallocatechin Monogallate; 2-Aminoethyldiphenyl Boronate; 2-Isopropyl-3-Methoxycinnamic Acid; 4,4′-diisothiocyanostilbene-2,2′-disulfonic acid Sodium Salt; 5-Chloroindole-2-Carboxylic Acid; 5-Fluoroindole-2-Carboxylic Acid; Acetohydroxamic Acid; Acetyl Tyrosine Ethyl Ester; Almotriptan; Almotriptan; Apramycin; Atorvastatin Calcium; Aurintricarboxylic Acid; Aurothioglucose; Butamben; Calcein; Crustecdysone; 2,3,4,6-tetrakis-O-(3-nitropropanoyl)-alpha-L-galactopyranose; Epicatechin Monogallate; Epigallocatechin-3-Monogallate; Glyburide; Hematein; Hinokitiol; Homosalate; Hydroquinone; Indole-2-Carboxylic Acid; Iopanic Acid; Irigenol; iriginol hexaaceatate; Levodopa; Lithium Citrate; Lovastatin; Meclizine Hydrochloride; Miglitol; N-(9-Fluorenylmethoxycarbonyl)-L-Leucine; Ononetin; Oxaprozin; Oxybenzone; Perillic Acid (−); Perindopril Erbumine; Prazosin Hydrochloride; Prenyletin; Quercetin Pentamethyl Ether; Ribostamycin Sulfate; Robustic Acid; Sparteine Sulfate; Sulconazole Nitrate; Sumaresinolic Acid; Suramin; Tannic Acid; Theaflavin; Theaflavin Digallate; or a pharmaceutically acceptable salt of any such compound, wherein the cellular uptake of proNGF is decreased at least about 60% than in the absence of the compound, when measured under same conditions.

2. The method according to claim 1, wherein the compound is Hydroquinone; Tannic Acid; Epigallocatechin-3-Monogallate; Aurintricarboxylic Acid; Sulconazole Nitrate; Levodopa; 2′,2′-Bisepigallocatechin Monogallate; 4,4′-diisothiocyanostilbene-2,2′-disulfonic acid Sodium Salt; Hematein; 5-Fluoroindole-2-Carboxylic Acid; Epicatechin Monogallate; Meclizine Hydrochloride; Theaflavin Digallate; Irigenol; Suramin; Atorvastatin Calcium; Indole-2-Carboxylic Acid; Iopanic Acid; N-(9-Fluorenylmethoxycarbonyl)-L-Leucine; Miglitol; Perillic Acid (−);Acetyl Tyrosine Ethyl Ester; Prenyletin; 2-Isopropyl-3-Methoxycinnamic Acid; Prazosin Hydrochloride; 5-Chloroindole-2-Carboxylic Acid; Theaflavin; Homosalate; Sumaresinolic Acid; Lovastatin; Robustic Acid; Glyburide; Oxybenzone; Calcein; 2,3,4,6-tetrakis-O-(3-nitropropanoyl)-alpha-L-galactopyranose; Sparteine Sulfate; iriginol hexaaceatate; 2-Aminoethyldiphenyl Boronate; Ribostamycin Sulfate; Hinokitiol; or a pharmaceutically acceptable salt of any such compound, and wherein the cellular uptake of proNGF is decreased at least about 70% than in the absence of the compound, when measured under same conditions.

3. The method according to claim 1, wherein the compound is Hydroquinone; Tannic Acid; Epigallocatechin-3-Monogallate; Aurintricarboxylic Acid; Sulconazole Nitrate; Levodopa; 2′,2′-Bisepigallocatechin Monogallate; 4,4′-diisothiocyanostilbene-2,2′-disulfonic acid Sodium Salt; Hematein; 5-Fluoroindole-2-Carboxylic Acid; Epicatechin Monogallate; Meclizine Hydrochloride; Theaflavin Digallate; Irigenol; Suramin; or a pharmaceutically acceptable salt of any such compound, and wherein the cellular uptake of proNGF is decreased at least about 85% than in the absence of the compound, when measured under same conditions.

4. The method according to claim 1, wherein the compound is Levodopa; lithium, or a statin such as lovastatin or a pharmaceutically acceptable salt of any such compound or a pharmaceutically acceptable salt thereof.

5. The method according to claim 4, wherein the mammal has suffered or is suffering from Alzheimer's disease.

6. The method according to claim 1, wherein the cell is a cell of the cardiovascular system.

7. The method according to claim 6, wherein the cell is an endothelial cell.

8. The method according to claim 6, wherein the cell is a smooth muscle cell.

9. The method according to claim 6, wherein the cell is a cardiomyocyte.

10. The method according to claim 1, wherein the mammal has suffered or is suffering from a cardiovascular disease or condition.

11. The method according to claim 10, wherein the cardiovascular disease or condition is any one of the following: atherosclerosis, acute coronary syndrome, transplantation-induced sclerosis, peripheral limb disease, intermittent claudication, diabetic complication, or thrombosis.

12. The method according to 11, wherein the acute coronary syndrome is any one of the following: unstable angina, thrombosis, myocardial infarction, plaque rupture, primary restinosis in a coronary artery, primary restinosis in a peripheral artery, secondary restinosis in a coronary artery, or secondary restinosis in a periphery artery.

13. The method according to 11, wherein the diabetic complication is any one of the following: ischemic heart disease, peripheral artery disease, congestive heart failure, retinopathy, neuropathy, or nephropathy.

14. The method according to claim 1, wherein the cell is a skin cell.

15. The method according to claim 14, wherein the skin cell is a hair follicle cell.

16. The method according to claim 14, wherein the skin cell is an immune system cell.

17. The method according to claim 14, wherein the skin cell is a keratinocyte.

18. The method according to claim 14, wherein the skin cell is a melanocyte.

19. The method according to claim 14, wherein the skin cell is a sebaceous gland cell.

20. The method according to claim 14, wherein the skin cell is a sweat gland cell.

21. The method according to claim 1, wherein the mammal has suffered from or is suffering from hair loss.

22. The method according to claim 1, wherein the mammal has suffered from or is suffering from hearing loss.

23. The method according to claim 1, wherein the cell is a neural cell.

24. The method according to claim 23, wherein the neural cell is a neuron.

25. The method according to claim 23, wherein the neural cell is a glial cell.

26. The method according to claim 1, wherein the mammal has suffered from or is suffering from trauma to the central nervous system, trauma to the peripheral nervous system, epilepsy-related brain damage, neurotoxic compound poisoning, radiation-induced brain damage, infectious disease of the central nervous system, infectious disease of the peripheral nervous system, stroke, ischemia, or neurodegenerative disease or condition.

27. The method according to claim 26, wherein the neurodegenerative disease or condition is any one of the following: Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Neuroborreliosis, Parkinson disease, Pelizaeus-Merzbacher Disease, Pick's disease, Primary lateral sclerosis, Prion diseases, Refsum's disease, Sandhoff disease, Schilder's disease, Schizophrenia, Spinocerebellar ataxia, Spinal muscular atrophy, Steele-Richardson-Olszewski disease, Tabes dorsalis, depression, psychiatric disorder, or dementia.

28. The method according to claim 1, wherein the mammal has suffered from or is suffering from cancer.

29. A method of promoting cellular uptake of pro-nerve growth factor (proNGF) in a cell expressing p75^{NTR} receptor in a mammal in need thereof, the method comprising administering to the mammal an effective amount of any one or any combination of the following promoting compounds Trichlormethine; 6,7-Dihydroxyflavone; Tangeritin; 3′,4′-Dihydroxyflavone; Amsacrine Hydrochloride; Putrescine Dihydrochloride; Flunixin Meglumine; 3-Benzyloxy-4,4-Bisnor-8,11,13-Podocarpatriene; 2-Acetylpyrrole; Dantron; 2,3,4-Trihydroxy-4′-Ethoxybenzophenone; 8-Cyclopentyltheophylline; Aminocyclopropanecarboxylic Acid; 2′,3-Dihydroxy-4,4′,6′-Trimethoxychalcone; 3,5-Dinitrocatechol (Or-486); Aztreonam; Piceid; Rifaximin; 10-Hydroxycamptothecin; 5,7-Dihydroxyflavone; ANTIMYCIN A; Xanthoxylin; or a pharmaceutically acceptable salt of any such compound, wherein the cellular uptake of proNGF is increased at least about 180% than in the absence of the compound, when measured under same conditions.

30. The method according to claim 29, wherein the compound is 10-Hydroxycamptothecin; 5,7-Dihydroxyflavone; ANTIMYCIN A; Xanthoxylin; or a pharmaceutically acceptable salt of any such compound, wherein the cellular uptake of proNGF is increased at least about 400% than in the absence of the compound, when measured under same conditions.

31. The method according to claim 29, wherein the compound is Xanthoxylin.
